(19) United States
(12) Patent Application Publication
(10) Pub. No.: US 2023/0233537 A1
(43) Pub. Date: Jul. 27, 2023

(54) Compositions and Kits of Parts Comprising N,N-Dimethyltryptamine and Harmine and Their Use in Therapy

(71) Applicant: University of Zürich, Zürich (CH)

(72) Inventors: Dario Dornbierer, Egg (CH); Davor Kosanic, Lausanne (CH); Milan Scheidegger, Winterthur (CH)

(21) Appl. No.: 18/012,122

(22) PCT Filed: Jun. 22, 2021
(86) PCT No.: PCT/EP2021/067051

§ 371 Date: Dec. 21, 2022

(30) Foreign Application Priority Data
Jun. 22, 2020 (EP) 20181489.4

(51) Int. Cl. A61K 31/437; A61K 31/4045; A61K 9/00; A61P 25/28

(52) U.S. Cl. CPC A61K 31/437; A61K 31/4045; A61K 9/0043; A61K 9/006; A61P 25/28

(57) ABSTRACT

The invention relates to a kit of parts comprising N,N-dimethyltryptamine or a pharmaceutically acceptable salt thereof, and a pharmaceutically acceptable carrier; and harmine or a pharmaceutically acceptable salt thereof, and a pharmaceutically acceptable carrier. Also provided is a composition comprising N,N-dimethyltryptamine fumarate and harmine hydrochloride. Further, the invention relates to a pharmaceutical composition comprising N,N-dimethyltryptamine or a pharmaceutically acceptable salt thereof, and a pharmaceutically acceptable carrier; and harmine or a pharmaceutically acceptable salt thereof, and a pharmaceutically acceptable carrier.

The invention relates to a kit of parts comprising N,N-dimethyltryptamine or a pharmaceutically acceptable salt thereof, and a pharmaceutically acceptable carrier; and harmine or a pharmaceutically acceptable salt thereof, and a pharmaceutically acceptable carrier. Also provided is a composition comprising N,N-dimethyltryptamine fumarate and harmine hydrochloride. Further, the invention relates to a pharmaceutical composition comprising N,N-dimethyltryptamine or a pharmaceutically acceptable salt thereof, and a pharmaceutically acceptable carrier; and harmine or a pharmaceutically acceptable salt thereof, and a pharmaceutically acceptable carrier.

Affective spectrum disorders are widespread in society and are significant contributors to the current economic burden in health care, reaching double-digit billion CHF amounts in Switzerland and orders of magnitude more worldwide. Along the affective spectrum the most prevalent mood disorders include depression (major depressive disorder, dysthymia, double depression, seasonal affective disorder, burnout, postpartum depression, premenstrual dysphoric disorder) and bipolar disorders (characterized by periods of depression and hypomania/mania). Despite high prevalence, most of the available therapies show suboptimal efficacy and are currently prescribed in a lengthy trial and error approach for weeks or months to see clinical benefit. Still fewer than 50% of all patients with depression show full remission with optimized standard treatment, including trials on numerous medications. Thus, there is an urgent need for novel depression therapies with more rapid and sustainable therapeutic effects.

Recently, a novel class of rapid-acting antidepressant psychotropic compounds such as ketamine, psilocybin, and LSD was discovered to alleviate symptoms of anxiety and depression. Repeated administration of ketamine was shown to sustain antidepressant effects, but puts patients at risk due to its addictive potential. Moreover, there are major shortcomings of using compounds such as LSD for clinical purposes due to its long duration of action (10-12 hours). Additionally, both LSD and psilocybin show rapid tolerance at serotonergic receptors (Nichols 2016), which makes them less suited for repeated dosing regimens.

In contrast, the traditional indigenous plant concoction commonly made from Banisteriopsis caapi and Psychotria viridis or Diplopterys cabrerana, called ayahuasca, known as psychedelic agent, is increasingly recognized due to its beneficial effects on physical and mental health, making it a promising candidate for therapeutic use (Dominguez-Clavé et al. 2016). Herein, psychedelic agent refers to an agent that can cause an altered state of consciousness in a subject that uses it. Altered state of consciousness refers to any condition different from a normal waking state, and may include, but is not limited to, experiencing cognitive or perceptual alterations (e.g. hallucinations), intense emotions, or day-dreaming. Ayahuasca has been suggested to exhibit positive effects in patients with psychological, somatic, and psychosomatic illnesses and has been used for centuries in natural medicine in Latin American regions (Frecska et al. 2016). In small pilot studies, ayahuasca shows rapid and more sustained antidepressant properties in depressed patients (Osório et al. 2015; Palhano-Fontes et al. 2018; Santos et al. 2016), compared to the transient antidepressant effects of ketamine, where a considerable number of patients relapse within 7 days of treatment (Sanacora et al. 2016). While the mechanism of such action is not known, the potentially therapeutic effect of ayahuasca is hypothesized to rely on its ability of resetting neuronal circuits underlying maladaptive neurobehavioral states.

Ayahuasca concoction comprises a mixture of N,N-dimethyltryptamine (DMT) and beta-carbolines (e.g. harmine, harmaline, tetrahydroharmine, among others.). Ayahuasca is a) non-toxic, b) has a low addictive abuse potential, c) does not produce tolerance, and d) shows an antidepressant potential (Dominguez-Clavé et al. 2016; Barbosa et al. 2012). In order for DMT to become bioavailable, peroral formulations usually contain plant-based sources of DMT (e.g. from Psychotria viridis) combined with β-carbolines (e.g. from Banisteriopsis caapi) that act as selective reversible monoamine oxidase A (MAO-A) inhibitors to prevent degradation of DMT in the body (Callaway et al. 1996). DMT is a structural analogue of serotonin and is widely found in nature, including plants, mammalian organisms, human brains and body fluids (Barker 2018).

N,N-Dimethyltryptamine and harmine (7-Methoxy-1-methyl-9H-pyrido[3,4-b]indol) may be represented by the chemical formulae shown below:

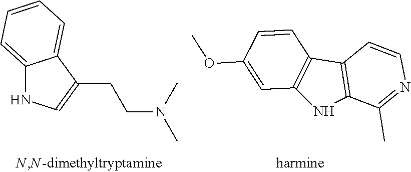

It has been suggested that ayahuasca can be potentially useful in treating a number of disorders, including depression and anxiety disorders. For example, in 2018 it was reported that a single dose of ayahuasca significantly reduced symptoms of treatment-resistant depression in a small placebo-controlled trial (Palhano-Fontes et al. 2018). Another preliminary study reports statistically significant reductions of up to 82% in depressive scores between baseline and 1, 7, and 21 days after ayahuasca administration, as measured on the Hamilton Rating Scale for Depression (HAM-D), the Montgomery-Asberg Depression Rating Scale (MADRS), and the Anxious-Depression subscale of the Brief Psychiatric Rating Scale (BPRS) (Osório et al. 2015).

Although ayahuasca ingestion is considered safe (Barbosa et al. 2012), it brings along a number of undesired side effects (e.g. nausea, vomiting, diarrhea, hallucinations), compromising its clinical utility. Most of its side effects can be attributed to suboptimal pharmacokinetic/-dynamic properties due to the random admixture of plant material (with unknown or adverse toxicity), and variability in alkaloid content—precluding its use as a standardized prescription medicine in the clinical context. Moreover, upon peroral administration of ayahuasca, DMT is readily absorbed into the bloodstream and causes rapid changes in the consumer's perception, with potentially distressing side effects (Riba et al. 2003).

An alternative to ayahuasca that solves the problems of side effects outlined above is pharmahuasca, also known as synthetic ayahuasca. According to the disclosure of the document DE102016014603A1, the term pharmahuasca or synthetic ayahuasca relates to combinations, compositions, mixtures and preparations comprising at least two members of the group of the active ingredients naturally occurring in and isolatable from Banisteriopsis caapi, Psychotria viridis and/or Diplopterys cabrerana, consisting of harmine, harmaline, d-tetrahydroharmine, N,N-dimethyltryptamine (DMT), mono-N-methyltryptamine, 5-methoxy-N,N-dimethyltryptamine, 5-hydroxy-N,N-dimethyltryptamine, 2-methyl-1,2,3,4-tetrahydro-β-carboline, harmol, harmalol, tetrahydroharmol, as well as their natural and unnatural stereoisomers and racemates, available in solid, liquid or semi-solid form, characterized in that at least one of the active ingredients is selected from the group of β-carbolines consisting of harmine, harminole and tetrahydrohamine and its stereoisomers and racemate, and at least one of the active substances is selected from the group containing terminal N-substituted tryptamines consisting of N,N-dimethyltryptamine (DMT), mono-N-methyltryptamine, 5-methoxy-N,N-dimethyltryptamine, 5-hydroxy-N,N-dimethyltryptamine. The active ingredients can be contained independently, in whole or in part—individually and in admixture, together and in several dosage forms, in the form of bases or their natural and synthetic salts, where applicable, or as N-oxides, bound to ion exchangers or another matrix, can be present as complexes and inclusion compounds, can be synthesized and/or can be obtained from any natural plant material by extraction and—the sum of the concentrations of the active ingredients is at least 0.0001%. It was hypothesized that peroral pharmahuasca would be more tolerable compared to traditional ayahuasca, due to the elimination of plant admixtures with unknown toxicity, which are known to cause undesired side effects (e.g. vomiting, nausea, diarrhea). According to Wikipedia (https://en.wikipedia.org/wiki/Pharmahuasca), 50 mg DMT and 100 mg harmaline is usually the recommended dosage per person for pharmahuasca. However, combinations of 50 mg harmaline, 50 mg harmine, and 50 mg DMT have been tested with success. The constituents are put into separate gelatin capsules. The capsule with harmaline/harmine is swallowed first and the capsule containing the DMT is taken 15 to 20 minutes later.

It will be however noted that while pharmahuasca compositions are known in the state of the art, medical use of pharmahuasca has not been demonstrated up to date. Clinical safety and efficacy of psychedelic drugs, e.g. DMT, crucially depend on several factors including the choice of the adequate dose, the patient's psychological expectations towards the psychedelic experience and professional psychotherapeutic guidance throughout the whole process of psychedelic therapy. In addition to that, the pharmacokinetic profile of a psychedelic drug substantially influences the dynamics of its psychological effects. If the absorption of DMT into the bloodstream follows a rapid kinetic—e.g. after intravenous, inhalative, or perorally-activated administration (combined with monoamine oxidase inhibitors, e.g. MAO-A inhibitors)—subjects initially often report intense subjective effects including disorientation accompanied by hallucinations and anxiety (Strassman et al. 1994; Riba et al. 2003; Riba et al. 2015). It is therefore necessary to reduce undesired side effects (e.g. elementary hallucinations, anxiety, dissociation, nausea, vomiting, and other physical side effects), while maximizing therapeutic efficacy, increasing the safety profile and ensuring the administration compliance in a clinical setting.

The underlying technical problem is thus the provision of pharmahuasca in a form suitable for treating a psychiatric, psychosomatic and/or somatic disorder in a clinical setting to provide a long term effect with reduced side effects.

The technical problem is solved by the embodiments presented herein and as characterized in the claims.

In a first aspect, the present invention relates to a kit of parts comprising (a) N,N-dimethyltryptamine or a pharmaceutically acceptable salt thereof, and a pharmaceutically acceptable carrier; and (b) harmine or a pharmaceutically acceptable salt thereof, and a pharmaceutically acceptable carrier.

In a second aspect, the present invention relates to a pharmaceutical composition comprising: (a) N,N-dimethyltryptamine or a pharmaceutically acceptable salt thereof, and a pharmaceutically acceptable carrier; and (b) harmine or a pharmaceutically acceptable salt thereof, and a pharmaceutically acceptable carrier.

Compounds referred to herein or pharmaceutically acceptable salts thereof may exist as hydrates, solvates and polymorphs thereof. The term “polymorphs” refers to the various crystalline structures of the compounds of the present invention. This may include, but is not limited to, crystal morphologies (and amorphous materials) and all crystal lattice forms. Salts of the present invention can be crystalline and may exist as more than one polymorph. Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

Based on the shortcomings of traditional ayahuasca, the present inventors have performed an open-label dose-finding pilot study in 10 healthy male volunteers to investigate PK/PD profiles and the tolerability of perorally administered pharmahuasca. Surprisingly, as shown in the Examples, the present inventors have found interindividual differences in plasma concentrations of both DMT and harmine after a single peroral administration, making it unsuitable for a reproducible clinical use. Thereby, peak plasma concentrations (also referred to as Cₘₐₓ) varied substantially by a factor of about 7 for DMT and by a factor of about 50 for harmine across subjects (see Example 1 for details and FIGS. 1 and 2 for an example). Likewise, subjective drug effects varied dramatically between subjects, with peak intensity ratings ranging from 1 (non-responder) to 10 (responder). This considerable difference in interindividual responsiveness was highly unexpected and raised doubts, whether pharmahuasca would be clinically applicable.

Surprisingly, the present inventors have found that the problems with interindividual differences in plasma concentrations of DMT and harmine can be overcome if DMT and harmine are administered so that the gastrointestinal tract is avoided (in other words, wherein DMT and harmine are not administered perorally). As demonstrated in the second open-label dose-response pilot study in the same 10 volunteers (see Example 2 for details), the non-peroral administration of DMT and harmine is superior to peroral pharmahuasca or traditional ayahuasca, in terms of response predictability, safety, tolerability and overall therapeutic efficacy.

Subjective drug effects of the psychedelic drugs, in the present invention of DMT, are herein defined and assessed by visual analogue scales, also referred to as VAS (e.g. intensity, liking, distress, arousal, relaxation, anxiety, loss of control, visual effects) and the following well-established psychometric tools including the Altered States of Consciousness Rating Scale (5D-ASC), Positive and Negative Affect Schedule (PANAS), Karolinska Sleepiness Scale (KSS), MINDSENS composite index, Nature Relatedness Scale, Cognitive Flexibility Inventory, Acceptance and Action Questionnaire II, Persisting Effects Questionnaire (PEQ), Challenging Experiences Questionnaire, Psychological Well-Being Post-Traumatic Changes Questionnaire (PWB-PTCQ), and the Symptom Checklist (SCL-90-R). Two additional psychometric tools can be used that are currently under validation by the research group of Prof. R. Carhart-Harris (Imperial College London): Connectedness questionnaire (CQ), and the Emotional breakthrough inventory (EBI). Preferably, the well-established psychometric tools as discussed herein include Altered States of Consciousness Rating Scale (5D-ASC), Positive and Negative Affect Schedule (PANAS), Connectedness questionnaire (CQ), and the Emotional breakthrough inventory (EBI).

Preferably, visual analogue scales (VAS) of general drug effects (e.g. intensity, liking, distress, arousal, relaxation, anxiety, loss of control, visual effects) and the Altered States of Consciousness Rating Scale (5D-ASC) (Studerus et al. 2010) are used in the present invention to assess subjective drug effects. Further preferred VAS are intensity, valence, side effects. The 5D-ASC assessment may comprise the assessment of the following parameters: oceanic boundlessness (e.g. positively experienced ego dissolution), anxious ego-disolution, visionary restructuralization, audio-visual changes, and/or vigilance reduction. Additional subdimensions of the ASC questionnaire capture experiential categories such as blissful state, spiritual experience, experience of unity, changed meaning of percepts, audio-visual synesthesia, complex imagery, elementary imagery, anxiety, impaired cognition and control, disembodiment, and insightfulness. These parameters are assessed individually by each subject by answering questions from the 5D-ASC questionnaire (validated for retrospective assessments) or by indicating their state of consciousness while under the effects of the drug (e.g. by visual analogue scales queried at regular intervals during the psychedelic session). It is known to the skilled person that subjects that use psychedelic substances feel the need to talk about their experience under the influence of a psychedelic substance while the effect of the drug decreases. Therefore, additional information on subjective drug effects can typically and preferably be obtained by semi-structured and audio-recorded psychological and/or phenomenological interviews according to standard established procedures known to a skilled person.

Safety and tolerability of the combined use of DMT and harmine can be increased by reducing the side effects associated with the use of DMT and/or harmine. Common somatic side effects include nausea, vomiting and diarrhea. By bypassing the GI tract according to the methods of the present invention, reduced stimulation of intestinal 5-HT3 chemoreceptors on the terminals of vagal afferents is achieved, yielding less nausea, less vomiting and less gastrointestinal discomfort. It is known to the skilled person that reduced nausea improves overall controllability of the drug effects, as less substance is being lost through vomiting. Therefore, there is no need for dose substitution. It should also be noted that co-ingestion of peroral harmine and tyramine-containing dietary products can lead to hypertensive crises, due to tyramine-mediated sympathetic nerve stimulation. Thus, circumventing the GI tract reduces the risk of dietary incompatibilities/interactions, and there is no or reduced need for fasting or special dietary requirements before drug administration.

Traditional (herbal) ayahuasca contains a plant matrix that frequently causes diarrhea in subjects that ingest it, due to the organoleptic properties of the decoction. These adverse effects are eliminated by circumventing the GI tract according to the present invention. Further, a reduced load of herbal admixtures with unfavorable toxicological profile (e.g. conveyed by the presence of harmaline) or other compounds causing physical discomfort (e.g. tannins) is achieved according to the methods of the present invention.

Further side effects of traditional ayahuasca or of pharmahuasca according to the state of the art include psychophysiological distress during the initial phase of drug action. To this end incremental administration of DMT according to the present invention flattens the peak of plasma concentration of DMT. The peak of plasma concentration of DMT, as depicted in FIG. 8A, can be associated with strong hallucinatory phenomena, anxiety or disorientation. Furthermore, due to short half-life of DMT, incremental administration prevents accumulation of DMT (see FIG. 8B) which can otherwise lead to strong and prolonged periods of hallucinatory phenomena, anxiety or disorientation (as seen for example for LSD, psilocybin, mescaline) with adverse consequences for the patient. Incremental DMT administration further allows for interruption of the treatment in case of adverse mental or physical effects. Further, a therapeutic window can be safely achieved by incremental dosing (no initial distressing effects of a high bolus dose).

In the present invention, the kit of parts refers to a combination of individual components (a) and (b) which are kept physically separate but adjacent. The skilled person will understand that the components (parts) of the kit may be combined before administration, that the components (parts) may be administered simultaneously, or that the components (parts) of the kit may be administered sequentially. In the case of sequential administration, the components (parts) of the kit are typically to be administered preferably within a time range of between 30 minutes and 120 minutes in order to achieve the effects of the present invention. The components of the kit of parts can be formulated for different routes of administration. It is known to the skilled person that small molecule drugs can be administered through peroral route of administration, parenteral route of administration (including intravenous route of administration, intramuscular route of administration, and subcutaneous route of administration), nasal (or intranasal) route of administration, ocular route of administration, transmucosal route of administration (buccal route of administration, sublingual route of administration, vaginal route of administration, and rectal route of administration), transdermal route of administration, inhalation route of administration and transdermal route of administration. It should be noted that herein, oral route of administration may refer to peroral route of administration, buccal route of administration and/or sublingual route of administration. Herein, components (a) and (b) may be formulated for administration through any of these routes of administration. It will be understood that (a) and (b) can be formulated for administration using the same route of administration, it will be further understood that (a) and (b) can be formulated for administration using different routes of administration.

According to the present invention, in the pharmaceutical composition comprising (a) and (b), (a) and (b) will be mixed together or packaged together, being suitable for being administered together. It is known to the skilled person that small molecule drugs can be administered through peroral route of administration, parenteral route of administration (including intravenous route of administration, intramuscular route of administration, and subcutaneous route of administration), nasal (or intranasal) route of administration, ocular route of administration, transmucosal route of administration (buccal route of administration, sublingual route of administration, vaginal route of administration, and rectal route of administration), inhalation route of administration and transdermal route of administration. Herein, if (a) and (b) are comprised within one composition, they are typically formulated for the same route of administration.

The dosage will depend on the route of administration, the severity of the disease, age and weight of the subject and other factors normally considered by the attending physician, when determining the individual regimen and dosage level for a particular patient or subject. The parts of the kit of parts or the pharmaceutical composition of the present invention may be administered via any route, including parenteral, intramuscular, subcutaneous, topical, transdermal, intranasal, intravenous, sublingual or intrarectal administration.

The parts of the kit of parts of the invention or the pharmaceutical composition of the invention may be prepared by mixing suitably selected and pharmaceutically acceptable excipients, vehicles, adjuvants, additives, surfactants, desiccants or diluents known to those well-skilled in the art, and can be suitably adapted for peroral, transmucosal, parenteral or topical administration. Typically and preferably the parts of the kit or the pharmaceutical composition of the invention are administered in the form of a tablet, orodispersible tablet, mucoadhesive film, lyophilizates, capsule, sachets, powder, granule, pellet, peroral or parenteral solution, suspension, suppository, ointment, cream, lotion, gel, paste and/or may contain liposomes, micelles and/or microspheres.

The term “pharmaceutically acceptable” indicates that the compound or composition, typically and preferably the salt or carrier, must be compatible chemically or toxicologically with the other ingredient(s), typically and preferably with the inventive composition or with the parts of the inventive kit of parts, when typically and preferably used in a formulation or when typically and preferably used for treating the animal, preferably the human, therewith. Preferably, the term “pharmaceutically acceptable” indicates that the compound or composition, typically and preferably the salt or carrier, must be compatible chemically and toxicologically with the other ingredient(s), typically and preferably with the inventive composition or with the parts of the inventive kit of parts, when typically and preferably used in a formulation or when typically and preferably used for treating the animal, preferably the human, therewith. It is noted that pharmaceutical compositions can be formulated by techniques known to the person skilled in the art, such as the techniques published in “Remington: The Science and Practice of Pharmacy”, Pharmaceutical Press, 22^{nd} edition.

The pharmaceutically acceptable carrier of the parts (a) and (b) of the kit of parts of the present invention or of the pharmaceutical composition of the present invention is without limitation any pharmaceutically acceptable excipient, vehicle, adjuvant, additive, surfactant, desiccant or diluent. Suitable pharmaceutically acceptable carriers are magnesium carbonate, magnesium stearate, talc, lactose, sugar, pectin, dextrin, starch, tragacanth, methyl cellulose, hydroxy-propyl-methyl-cellulose, sodium carboxymethyl cellulose, a low-melting wax, cocoa butter. Pharmaceutically acceptable carriers of the invention can be solid, semi-solid or liquid.

Tablets, capsules or sachets for peroral administration are usually supplied in dosage units and may contain conventional excipients, such as binders, fillers, diluents, tableting agents, lubricants, detergents, disintegrants, colorants, flavors and wetting agents. Tablets may be coated in accordance to methods well known in the art. Suitable fillers include or are preferably cellulose, mannitol, lactose and similar agents. Suitable disintegrants include or are preferably starch, polyvinyl pyrrolidone and starch derivatives such as sodium starch glycolate. Suitable lubricants include or are preferably, for example, magnesium stearate. Suitable wetting agents include or are preferably sodium lauryl sulfate. These solid oral compositions can be prepared with conventional mixing, filling or tableting methods. The mixing operations can be repeated to disperse the active agent in compositions containing large quantities of fillers. These operations are conventional.

The parts of the kit of parts of the invention may be prepared by mixing suitably selected and pharmaceutically acceptable excipients, vehicles, adjuvants, additives, surfactants, desiccants or diluents known to those well-skilled in the art, and can be suitably adapted for oral, parenteral or topical administration. Typically and preferably the parts of the kit of parts of the invention is administered in the form of a tablet, capsule, sachets, powder, granule, pellet, orodispersible tablet, mucoadhesive film, lyophilizate, oral or parenteral solution, suspension, suppository, ointment, cream, lotion, gel, paste and/or may contain liposomes, micelles and/or microspheres.

The parts of the kit of parts or the pharmaceutical composition of the present invention as liquid compositions for oral administration can be provided in the form of, for example, aqueous solutions, emulsions, syrups or elixirs or in the form of a dry product to be reconstituted with water or with a suitable liquid carrier at the time of use. The liquid compositions can contain conventional additives, such as suspending agents, for example sorbitol, syrup, methylcellulose, gelatin, hydroxyethyl cellulose, carboxymethyl cellulose, aluminum stearate gel or hydrogenated edible fats; emulsifying agents, for example lecithin, sorbitan monooleate, or acacia; non aqueous carriers (which can include edible oil), for example almond oil, fractionated coconut oil, oily esters, such as glycerin esters, propylene glycol or ethyl alcohol; preservatives, for example methyl or propyl p-hydroxybenzoate or sorbic acid; penetration enhancer, for example dimethylsulfoxide (DMSO); pH buffer systems, for example phosphate buffer, carbonate buffer, citrate buffer, citrate-phosphate buffer and other pharmaceutically acceptable buffer systems; solubilizers, for example beta-cyclodextrin, and if desired, conventional flavors or colorants. Oral formulations may also include or may be formulated as conventional formulations, such as tablets or granules.

Oral formulations may optionally further include taste-masking components to optimize the taste) perception of the oral formulation. Examples of such taste-masking components may be citrus-, licorice-, mint-, grape-, black currant- or eucalyptus-based flavorants known to those well-skilled in the art.

The form of dosage for intranasal administration may include solutions, suspensions or emulsions of the active compound in a liquid carrier in the form of nose drops. Suitable liquid carriers include water, propylene glycol and other pharmaceutically acceptable alcohols. For administration in drop form formulations may suitably be put in a container provided e.g. with a conventional dropper/closure device, e.g. comprising a pipette or the like, preferably delivering a substantially fixed volume of composition/drop. The dosage forms may be sterilized, as required. The dosage forms may also contain adjuvants such as preservatives, stabilizers, emulsifiers or suspending agents, wetting agents, salts for varying the osmotic pressure or buffers, as required. Buffer systems may include for example phosphate buffer, carbonate buffer, citrate buffer, citrate-phosphate buffer and other pharmaceutically acceptable buffer systems. Intranasal formulations may optionally further include smell-masking components to optimize the smell.

For parenteral administration, liquid dosage units can be prepared containing the inventive composition and a sterile carrier, or the parts of the inventive kit of parts, and a sterile carrier. The parenteral solutions are normally prepared by dissolving the compound in a carrier and sterilizing by filtration, autoclavation, before filling suitable vials or ampoules and sealing.

Adjuvants, such as local anesthetics, preservatives and buffering agents can be added to the pharmaceutical composition or to the parts of the kit of parts of the present invention. In order to increase stability, the pharmaceutical composition or the parts of the kit of parts can be frozen after filling the vial and the water can be removed under vacuum. A surfactant or humectant can be advantageously included in the pharmaceutical composition or in the parts of the kit of parts in order to facilitate uniform distribution of the inventive composition or the parts of the inventive kit of parts.

Topical formulations include or are preferably ointments, creams, lotions, gels, gums, solutions, pastes or may contain liposomes, micelles or microspheres.

The term “preferably” is used to describe features or embodiments which are not required in the present invention but may lead to improved technical effects and are thus desirable but not essential.

With respect to the numerical values mentioned herein, unless explicitly stated otherwise, the last decimal place of a numerical value preferably indicates its degree of accuracy. Thus, unless other error margins are given, the maximum margin is preferably ascertained by applying the rounding-off convention to the last decimal place. Thus, a value of 2.5 preferably has an error margin of 2.45 to 2.54.

DMT (N,N-dimethyltryptamine) is a psychedelic substance that is a structural analogue of serotonin and melatonin. DMT is also a structural and functional analogue of other psychedelic substances, including bufotenin (5-hydroxy-N,N-dimethyltryptamine), psilocybin (phosphate ester of 4-hydroxy-N,N-dimethyltryptamine) and psilocin (4-hydroxy-N,N-dimethyltryptamine). Further known analogues of DMT include mono-N-methyltryptamine. The analogues of DMT listed herein also show activity as psychedelic agents. Furthermore, the analogues of DMT listed herein are all mono-amines, and as such are potential substrates of MAO-A monoamine oxidase. Therefore, it is further envisaged that psilocybin, psilocin, and mono-N-methyltryptamine may also be used in the compositions, the pharmaceutical compositions, the kits of parts and/or the methods of the present invention, replacing DMT. In particular, it is envisaged that psilocin or psilocybin may be used in the compositions, the pharmaceutical compositions, the kits of parts and/or the methods of the present invention, replacing DMT. Such a pharmaceutical composition, composition or kit of parts can also be referred to as psilohuasca. It is conceivable to the skilled person, that such pharmaceutical composition, composition or kit of parts comprising psilocin or psilocybin instead of DMT could be used to treat the diseases that can be treated with the pharmaceutical compositions, compositions or the kits of parts of the present invention.

Harmine (7-methoxy-1-methyl-9H-pyrido[3, 4-b]-indole), also known as banisterine or as telepathine, is an alkaloid that occurs in a number of different plants, including harmel (Peganum harmala) or Banisteriopsis caapi. It belongs to a group of beta-carbolines. Harmine reversibly inhibits monoamine oxidase A (MAO-A), but it does not inhibit the monoamine oxidase B (MAO-B). In the compositions, the pharmaceutical compositions, the kits of parts and the methods of the present invention harmine is used as an inhibitor of MAO-A. Several structural analogues of harmine include harmaline, tetrohydroharmine, harmol, harmalol, tetrahydroharmol, 2-methyl-1,2,3,4-tetra-hydro-β-carboline. It is noted that the analogues of harmine listed herein are all MAO-A inhibitors. Therefore, it is further envisaged that harmaline, tetrohydroharmine, harmol, haramolol, tetrahydroharmol, 2-methyl-1,2,3,4.tetra-hydro-β-carboline may also be used in the compositions, the pharmaceutical compositions, the kits of parts and/or the methods of the present invention, replacing harmine.

Further, it is envisaged that compositions, pharmaceutical compositions or kits of parts, instead of harmine as monoamine oxidase inhibitor may include another available inhibitor. For example moclobemid (4-chloro-N-(2-morpholino-ethyl)benzamide), commercially known as Aurorix®, can be used for this purpose. Further non-limiting examples of selective MAO-A inhibitors that can be used within the scope of the present invention include bifemelane (4-(O-benzylphenoxy)-N-methylbutylamine), pirindole, toloxatone and minaprine. As known to the skilled person, curcumin can be used as a reversible inhibitor of MAO-A.

According to the present invention, in the pharmaceutical composition of the present invention or in the kit of parts of the present invention, (a) and (b) may include DMT and harmine, respectively, in their basic form or as pharmaceutically acceptable salts. It is known to the skilled person that the pharmaceutically acceptable acid addition salts are those formed from acids which form non-toxic acid anions such as the hydrochloride, hydrobromide, sulphate, phosphate or acid phosphate, acetate, maleate, fumarate, lactate, tartrate, citrate, oxalate, ascorbate and gluconate salts. However, this list is not meant to be limiting and any pharmaceutically acceptable salt can be used in the present invention.

In the present invention, DMT is preferably used as acetate salt, sulfate salt, oxalate salt, citrate salt, fumarate salt, ascorbate salt. Most preferably, DMT is used as a fumarate salt. DMT fumarate shows good aqueous solubility (typically >35 mg/mL at 298 K) and is suitable for mucosal absorption, and therefore is suitable for intranasal administration. It should be noted that compared with DMT in its basic form, DMT fumarate shows lower buccal absorption. DMT fumarate, as understood herein, refers preferably to a hemifumarate salt of DMT, which also may be understood as a salt wherein the molar ratio of DMT to fumarate is about 2:1, more preferably 2:1. It should be however noted that the use of DMT in its basic form is substantially limited by the burning sensation caused by basic corrosion of the oral mucosa, when administered buccally.

In the present invention, harmine can be used in its basic form or as its hydrochloride, acetate, fumarate, sulfate or citrate. It should be noted that solubility of harmine free base is very low. Most harmine salts, including hydrochloride, acetate, fumarate, sulfate and citrate, show low aqueous solubility (typically not more than 35 mg/mL at 298 K). Furthermore, when administered intranasally some of these salts lead to a burning sensation reported by subjects due to acidic corrosion of the nasal mucosa. In the present invention, harmine is preferably used as a free base or as a hydrochloride salt. Most preferably, harmine is used as a hydrochloride salt.

Therefore, in a further aspect, the kit of parts of the present invention and the pharmaceutical composition of the present invention relate to an embodiment, wherein (a) is N,N-dimethyltryptamine fumarate and a pharmaceutically acceptable carrier. In a yet further aspect, the kit of parts of the present invention and the pharmaceutical composition of the present invention relate to an embodiment, wherein (b) is harmine hydrochloride and a pharmaceutically acceptable carrier.

The compositions, pharmaceutical compositions, kits of parts and methods of the present invention relate to combinations or combined use of DMT and harmine. Therefore, any combination of DMT salts listed herein or its basic form and of harmine salt or its basic form can be used in the present invention. The kit of parts of the present invention or the pharmaceutical composition of the present invention may therefore comprise N,N-dimethyltryptamine in its basic form and harmine in a form selected from its basic form, hydrochloride, acetate, fumarate, sulfate, and citrate. The kit of parts of the present invention or the pharmaceutical composition of the present invention may alternatively comprise N,N-dimethyltryptamine as an acetate salt, and harmine in a form selected from its basic form, hydrochloride, acetate, fumarate, sulfate, and citrate. Alternatively, the kit of parts of the present invention or the pharmaceutical composition of the present invention may comprise N,N-dimethyltryptamine as a sulfate salt, and harmine in a form selected from its basic form, hydrochloride, acetate, fumarate, sulfate, and citrate. Alternatively, the kit of parts of the present invention or the pharmaceutical composition of the present invention may comprise N,N-dimethyltryptamine as an oxalate salt, and harmine in a form selected from its basic form, hydrochloride, acetate, fumarate, sulfate, and citrate. As a further alternative, the kit of parts of the present invention or the pharmaceutical composition of the present invention may comprise N,N-dimethyltryptamine as a citrate salt, and harmine in a form selected from its basic form, hydrochloride, acetate, fumarate, sulfate, and citrate. As a further alternative, the kit of parts of the present invention or the pharmaceutical composition of the present invention may comprise N,N-dimethyltryptamine as a fumarate salt, and harmine in a form selected from its basic form, hydrochloride, acetate, fumarate, sulfate, and citrate. Alternatively, the kit of parts of the present invention or the pharmaceutical composition of the present invention may comprise N,N-dimethyltryptamine as an ascorbate salt, and harmine in a form selected from its basic form, hydrochloride, acetate, fumarate, sulfate, and citrate.

It is further envisaged that in the kit of parts of the present invention or the pharmaceutical composition of the present invention N,N-dimethyltryptamine may be present in more than one form, which can be selected from a basic form, acetate salt, sulfate salt, oxalate salt, citrate salt, fumarate salt, and ascorbate salt. It is further envisaged that in the kit of parts of the present invention or the pharmaceutical composition of the present invention, harmine may be present in more than one form, selected from its basic form, hydrochloride, acetate, fumarate, sulfate, and citrate.

Preferably, in the kit of parts of the present invention or the pharmaceutical composition of the present invention N,N-dimethyltryptamine is used as a fumarate salt, and/or harmine is used as a hydrochloride. Most preferably, in the kit of parts of the present invention or the pharmaceutical composition of the present invention N,N-dimethyltryptamine is used as a fumarate salt, and harmine is used as a hydrochloride.

In a further aspect, the present invention relates to a composition comprising N,N-dimethyltryptamine fumarate and harmine hydrochloride. The composition comprising N,N-dimethyltryptamine fumarate and harmine hydrochloride, when combined with a pharmaceutically acceptable carrier, as defined herein, can be used to formulate a pharmaceutical composition according to the present invention.

In again a further aspect, the invention relates to the kit of parts of the present invention or the pharmaceutical composition of the present invention for use as a medicament.

In again a further aspect, the invention relates to the kit of parts for use of the present invention or the pharmaceutical composition for use of the present invention, for use in treating a psychiatric, psychosomatic, or somatic disorder. In again a further aspect, the present invention relates to the kit of parts for use of the present invention or the pharmaceutical composition for use of the present invention, for use in the manufacture of the medicament for treating a psychiatric, psychosomatic, or somatic disorder.

In again a further aspect, the invention provides for the pharmaceutical composition of the invention or the kit of parts of the invention for use in the treatment or prevention of a large number of diseases and disorders. In a further aspect, the invention provides for the pharmaceutical composition of the invention or the kit of parts of the invention for use in the manufacture of a medicament for treatment or prevention of a large number of disease and disorders. The said diseases and disorders are preferably selected from the following:

a) treatment of depression, depressive episode, major depressive disorder, dysthymia, double depression, seasonal affective disorder, treatment-resistant depression, depressive episodes in bipolar disorder, postpartum depression, premenstrual dysphoric disorder, and/or stress-related affective disorders, e.g. burnout or depression in patients with chronic somatic disorders;

b) treatment of anxiety such as panic attacks, panic disorder, acute stress disorder, agoraphobia, generalized anxiety disorder, separation anxiety disorder, social phobia, specific phobia, substance-induced anxiety disorder; treatment of obsessive-compulsive disorder, treatment of post-traumatic stress disorder, treatment of attachment disorders; and/or treatment of attention deficit disorders, such as attention-deficit hyperactivity disorder (ADHD), autism and autism-spectrum disorders, and/or impulse control disorder;

c) treatment and prevention of substance-related and/or behavioral addictions (such as gambling, eating, digital media, exercise or shopping); treatment of substance addiction, drug dependence, tolerance, dependence or withdrawal from substances including alcohol, amphetamines, cannabis, cocaine, caffeine, stimulants, research chemicals, hallucinogens, inhalants, nicotine, opioids, GHB, dissociatives (including ketamine, phencyclidine), sedatives, hypnotics or anxiolytics; treatment of smoking addiction; and/or as an agent to aid quitting smoking,

d) as a support agent for psychotherapy and/or psychoanalysis;

e) as a diagnostic aid for dysfunctions, and/or mental and somatic disorders.

f) treatment of sexual dysfunction;

g) treatment of neuroses; and/or as an agent for inducing deep relaxation;

h) as an agent for pharmacological induction of meditative states;

i) treatment of tendency to aggressive behavior of the patient against himself and against other persons; and/or treatment of behavioral disorders and socially harmful behavior;

j) treatment of alexithymia; and/or improvement of mentalization and social skills (e.g. in attachment/developmental disorders, autism spectrum disorders);

k) stimulation of oxytocin release;

l) as agent for increasing the concentration of neurotransmitters in the central nervous system; as agent for increasing the concentration of serotonin in the central nervous system; and/or as agent for increasing the concentration of dopamine in the central nervous system;

m) as neuroprotective and neuroregenerative agent; as anti-aging agent, regenerative agent, prevention and treatment of signs of aging; protection from free radical damage; and/or protection and improvement of damage by ionizing radiation;

n) as appetite regulation agent; as weight loss agent; treatment and prevention of obesity; treatment of eating disorders; activation of lipid metabolism and physiological fat burning; activation of carbohydrate metabolism; activation of physiological glycogen combustion; treatment and prevention of diabetes; and/or treatment of insulin resistance;

o) treatment of inflammation; treatment of chronic low grade inflammation, treatment of autoimmune disorders, including rheumatoid arthritis, systemic lupus erythematosis, inflammatory bowel disease, multiple sclerosis, type 1 diabetes mellitus, Guillain-Barre syndrome, and/or psoriasis; and/or treatment of autoinflammatory diseases, including Crohn's disease, and/or Behcet's disease.

p) stimulation of immune response;

q) as an antineoplastic agent; treatment and/or prevention of cancer, abnormal cell growth and mutation

It is to be understood that the above list of diseases is only given as specific examples and is not to be interpreted as limiting the present invention. Among the above, the preferred one(s) are one or more selected from a), b), and c).

Thus, in again a further aspect, the invention relates to the kit of parts for use of the present invention or the pharmaceutical composition for use of the present invention, wherein the psychiatric disorder is depression, stress-related affective disorder, major depressive disorder, dysthymia, treatment-resistant depression, burnout, anxiety, post-traumatic stress disorder, addiction, eating disorder, or obsessive-compulsive disorder.

It has been shown in a randomized controlled trial that ayahuasca (as well as DMT analogues, e.g. psilocybin), exhibit rapid-acting and sustained antidepressant efficacy in treatment-resistant depression after a single administration. At the same time, no addictive abuse potential and no signs of acute or chronic toxicity in long-term users of psilocybin or ayahuasca were found (which is not the case for ketamine that is currently being used off-label for the treatment of treatment-resistant depression).

According to the present inventors, the interindividual differences in the plasma level of DMT as observed upon peroral administration of DMT (as demonstrated in Example 1), may be due to interindividual differences in gastrointestinal expression of the MAO-A enzyme. MAO-A is highly expressed in the endothelium in the gastrointestinal tract and can degrade DMT before it can be adsorbed into the blood. Co-administration of harmine or another MAO-A inhibitor with DMT is supposed to counteract this effect. However, the administered dose of harmine, may be not sufficient in the case of individuals with higher than average MAO-A expression level, and as a consequence reduced peroral bioavailability may be observed for such individuals.

Further, it will be noted that bioavailability of harmine may be different between different subjects administered with the same peroral dose of harmine (as demonstrated in Example 1). According to the present inventors, the interindividual differences in the plasma level of harmine may be due to interindividual differences in hepatic degradation of harmine. Harmine is mainly degraded by the hepatic enzyme CYP2D6. The expression of this enzyme varies across subjects, causing differences in harmine bioavailability. Insufficient harmine bioavailability causes a rapid degradation of systemic DMT by MAO-A enzymes expressed in the mitochondria of cerebral neurons. It is herein noted that the skilled person could have solved this problem by increasing the plasma concentration of harmine by administering more harmine to a subject. However it is not desired to increase the dose of harmine administered to the subject, as the probability of undesired side effects (high harmine doses in certain subjects may result in harmine intoxication leading to sedative, emetic, and/or cardiovascular side effects) would rise with increased dose of harmine, which is incompatible with clinical application. Therefore, increasing the dose of harmine would not solve the problem of the present invention.

Thus, in again a further aspect, the kit of parts for use of the present invention or the pharmaceutical composition for use of the present invention relates to an embodiment, wherein wherein (a) and (b) are not to be administered perorally.

Accordingly, the pharmaceutical composition of the present invention, in other words the pharmaceutical composition comprising (a) and (b), is preferably administered in such a way that it does not pass the esophagus. In other words, preferably the gastrointestinal tract is avoided in the administration of the pharmaceutical composition of the present invention. Thus, the pharmaceutical composition of the present invention, in other words the pharmaceutical composition comprising (a) and (b), is preferably administered through parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration. More preferably, the said pharmaceutical composition is administered through transmucosal route of administration (buccal route of administration, sublingual route of administration, vaginal route of administration, and rectal route of administration).

Further according to the present invention, the parts or components of the kit of parts of the present invention, in other words (a) and/or (b), are preferably administered in such a way that they do not pass the esophagus. In other words, preferably the gastrointestinal tract is avoided in the administration of the parts or components of the kit of parts of the present invention. Thus, the parts or components of the kit of parts of the present invention, in other words (a) and/or (b), are preferably administered through parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration. As disclosed herein, as in the kit of parts (a) and (b) are not required to be mixed and/or to be packaged together, (a) can be administered through a different administration route than (b). Thus, according to the present invention, (a) can be administered through the parenteral route of administration (including intravenous route of administration, intramuscular route of administration, and/or subcutaneous route of administration), and (b) can be administered through a route of administration selected from parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration. Further according to the present invention, (a) can be administered through nasal (or intranasal) route of administration, and (b) may be administered through a route of administration selected from parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration. Again further according to the present invention, (a) can be administered through ocular route of administration, and (b) may be administered through a route of administration selected from parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration. Again further according to the present invention, (a) can be administered through transmucosal route of administration (buccal route of administration, sublingual route of administration, vaginal route of administration, and rectal route of administration), and (b) may be administered through a route of administration selected from parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration. Again further according to the present invention, (a) can be administered through transdermal route of administration, and (b) may be administered through a route of administration selected from parenteral route of administration (including intravenous route of administration, intramuscular route of administration, and/or subcutaneous route of administration), nasal (or intranasal) route of administration, ocular route of administration, transmucosal route of administration (buccal route of administration, sublingual route of administration, vaginal route of administration, and rectal route of administration), and transdermal route of administration.

It should be noted that in the pharmaceutical composition of the present invention, (a) and (b) are to be administered together and simultaneously to a subject. It will be further noted that in the kit of parts of the present invention, (a) and (b) can be administered simultaneously or sequentially to a subject. In the former case, parts (a) and (b) of the kit of parts of the present invention can be mixed before their simultaneous administration to a subject. However, (a) and (b) may also be packaged separately and administered to a subject sequentially. Preferably, (b) is to be administered first to a subject, followed by the administration of (a). This way, as (b) comprises an inhibitor of MAO-A, namely harmine or a pharmaceutically acceptable salt thereof, by inhibiting MAO-A in advance of and during the administration of DMT comprised in (a), the bioavailability of DMT in a subject may be increased. Preferably, (a) is to be administered not more than 120 minutes after administration of (b). Further preferably, (a) is to be administered not earlier than 30 minutes after administration of (b). It is however noted that this administration regime is not limiting, and that depending on the subject different dosing regimes may be proposed. For example, if according to the dosing regime (a) was to be administered more than 120 minutes after administration of (b), a skilled person could decide to administer the subject with a further dose of (b), as required.

Therefore, in again further aspect, the kit of parts for use of the present invention or the pharmaceutical composition for use of the present invention relates to an embodiment, wherein (a) and (b) are to be administered simultaneously or sequentially. In a further aspect, the kit of parts for use of the present invention or the pharmaceutical composition for use of the present invention relate to an embodiment, wherein the administration of (b) is followed by the administration of (a).

The route of administration may depend on the exact salt form of DMT comprised in (a), and on the exact salt form of harmine comprised in (b). The present inventors have demonstrated that DMT fumarate is absorbed better than DMT in its basic form through the buccal route of administration. At the same time, the present inventors have demonstrated high solubility and efficient transmucosal absorption of DMT fumarate, and developed a DMT fumarate nasal spray for administration and/or incremental dosing of DMT fumarate. Preferably in the methods of the present invention, (a) is administered through the nasal (or intranasal) route of administration. Herein, nasal (or intranasal) route of administration of a drug is understood as a route of administration wherein the drug is insufflated or instilled into the nasal cavity, preferably onto nasal mucosa, in other words wherein the drug is administered into the nasal cavity, preferably onto nasal mucosa.

Therefore, in again a further aspect, the kit of parts for use of the present invention or the pharmaceutical composition for use of the present invention relates to an embodiment, wherein (a) is to be administered intranasally.

Intranasal administration of DMT (combined with harmine administration) can be accomplished incrementally in hubs, as disclosed herein, which allows for individual dose-titration, and hence allows for less distressing aspects of psychotropic drug effects (e.g. anxiety, disorientation, hallucinations), and mellow onset of empathogenic drug effects, which are preferable for clinical applications [FIG. 8]. Said incremental intranasal administration, also referred to as metered intranasal administration of DMT (combined with sublingually or buccally administered harmine) yields a biphasic action profile with mellow psychotropic effects during the initial phase, which is followed by an empathogenic plateau, which is dependent on the previous continuous incremental DMT administration. The inventors conclude that this dosing regimen is increasing the overall safety, tolerability, and flexibility of psychedelic therapy with pharmahuasca in clinical populations.

In a further aspect, the kit of parts for use of the present invention or the pharmaceutical composition for use of the present invention relates to an embodiment, wherein (b) is to be administered buccally and/or sublingually.

Herein, sublingual administration of a drug is defined as placing the drug under the tongue of a subject so it is absorbed through oral mucosa. Further herein, buccal administration of a drug is defined as placing the drug between the gums and the cheek of a subject so it is absorbed through oral mucosa.

The present inventors have found harmine to be less suited for nasal application compared to DMT, due to the intense burning sensation caused by all of the investigated harmine salts (HCl, acetate, sulphate, fumarate, citrate). A lyophilized dispersible tablet to deliver harmine over the transmucosal route was therefore developed by the present inventors. Preferably, harmine is used herein in the form of harmine hydrochloride. Thanks to avoiding the gastrointestinal administration tract (also referred to as GI tract), and hence the lack of first-pass metabolism, in the present invention the harmine hydrochloride dose can be reduced to 150 mg, compared to 250 mg in a typical administration of peroral pharmahuasca, for example administration of peroral pharmahuasca as shown in Example 1 (Reference Example).

It has been demonstrated by the present inventors that combined administration of sublingual harmine HCl and intranasal DMT fumarate yields the best outcome in terms of safety, tolerability, bioavailability, and controllability, and thus represents an exclusively favourable galenic route of administration for subsequent drug development and clinical trials in patients. Significantly lower harmine doses are needed for sufficient MAO-A-inhibition when administered sublingually, as compared to peroral administration. Intranasal administration of DMT, combined with sublingual administration of harmine, allows flexibility in adjusting the experiential properties of the drug combination to the individual needs of the patient as shown in Example 2.

It is also part of the invention to provide a method for the treatment of a disease or disorder, wherein a therapeutically effective amount of the pharmaceutical composition of the invention or the kit of the invention is administered to an animal, preferably human, in need thereof. The term “therapeutically effective amount” here refers to the amount of DMT-comprising component comprised in (a), and to the amount of harmine-comprising component comprised in (b), that are sufficient to modulate one or more of the symptoms of the condition or disease being treated, preferably between 10 mg and 100 mg of N,N-dimethyltryptamine per administration and preferably between 10 mg to 1000 mg of harmine per administration, more preferably between 75 mg to 300 mg of harmine per administration. It is furthermore also a part of the invention to provide a method for the prevention of a disease or disorder, wherein a therapeutically effective amount of the composition of the invention or the pharmaceutical composition of the invention or the kit of the invention is administered to an animal, preferably human, reasonably expected to be in need thereof. The term “therapeutically effective amount” here refers to that amount sufficient to modulate one or more of the expected symptoms of the condition or disease to be avoided, preferably between 10 mg and 100 mg of N,N-dimethyltryptamine per administration and preferably between 10 mg and 1000 mg of harmine per administration, more preferably between 75 mg to 300 mg of harmine per administration. In another preferred embodiment of the present invention, therapeutically effective amount refers to that amount sufficient to modulate one or more of the expected symptoms of the condition or disease to be avoided, preferably between 100 mg and 160 mg of N,N-dimethyltryptamine per administration and preferably between 10 mg and 600 mg of harmine per administration, more preferably between 75 mg to 300 mg of harmine per administration.

The N,N-dimethyltryptamine can be used in the pharmaceutical compositions of the present invention or the kits of parts of the present invention in an amount of more than 10 mg and not more than 25 mg per administration, or it can be used in an amount of more than 25 mg and not more than 50 mg per administration, or in an amount of more than 50 mg and not more than 75 mg, or in an amount of more than 75 mg and not more than 100 mg per administration. The harmine can be used in the pharmaceutical compositions of the present invention or the kits of parts of the present invention in an amount of more than 75 mg and not more than 150 mg per administration, or it can be used in an amount of more than 150 mg and not more than 225 mg per administration, or in an amount of more than 250 mg and not more than 300 mg.

Thus, the pharmaceutical compositions of the present invention or the kits of parts of the present invention can comprise more than 10 mg and not more than 25 mg per administration of N,N-dimethyltryptamine, and more than 75 mg and not more than 150 mg per administration of harmine, or more than 150 mg and not more than 225 mg per administration of harmine, or in an amount of more than 250 mg and not more than 300 mg of harmine. The pharmaceutical compositions of the present invention or the kits of parts of the present invention can alternatively comprise more than 25 mg and not more than 50 mg per administration of N,N-dimethyltryptamine, and more than 75 mg and not more than 150 mg per administration of harmine, or more than 150 mg and not more than 225 mg per administration of harmine, or in an amount of more than 250 mg and not more than 300 mg of harmine. The pharmaceutical compositions of the present invention or the kits of parts of the present invention can alternatively comprise more than 50 mg and not more than 75 mg per administration of N,N-dimethyltryptamine, and more than 75 mg and not more than 150 mg per administration of harmine, or more than 150 mg and not more than 225 mg per administration of harmine, or in an amount of more than 250 mg and not more than 300 mg of harmine. The pharmaceutical compositions of the present invention or the kits of parts of the present invention can alternatively comprise more than 75 mg and not more than 100 mg per administration of N,N-dimethyltryptamine, and more than 75 mg and not more than 150 mg per administration of harmine, or more than 150 mg and not more than 225 mg per administration of harmine, or in an amount of more than 250 mg and not more than 300 mg of harmine.

Thus, in a further aspect, the kit of parts for use of the present invention or the pharmaceutical composition for use of the present invention relates to an embodiment, wherein (a) is to be administered in a dose of between 10 mg to 100 mg of N,N-dimethyltryptamine per administration, preferably in an incremental manner over a period of time of between 60 to 180 minutes.

In a further preferred aspect, the kit of parts for use of the present invention or the pharmaceutical composition for use of the present invention relates to an embodiment, wherein (a) is to be administered in a dose of between 100 mg to 160 mg of N,N-dimethyltryptamine per administration, preferably in an incremental manner over a period of time of between 60 to 180 minutes.

In again a further aspect, the kit of parts for use of the present invention or the pharmaceutical composition for use of the present invention relates to an embodiment, wherein (b) is to be administered in a dose of between 75 mg to 300 mg of harmine per administration, more preferably 100 mg to 150 mg of harmine per administration.

According to the present invention, (a) can be administered to a subject substantially at once, or preferably it can be administered in an incremental manner over a certain period of time. By administering substantially at once, administration in one portion over a period of time of 1 minute or less is meant here. As a high dose (for example, 100 mg or more) of (a) administered at once (i.e., within less than one minute) would cause hallucinations and mental physical discomfort, it is preferable to administer high doses of (a) sequentially (e.g. 10 mg of (a) every 10-20 minutes). In other words, preferably between 10 mg and 100 mg of N,N-dimethyltryptamine can be administered to a subject in an incremental manner over a certain period of time, preferably less than 240 minutes (4 hours). More preferably, between 10 mg and 100 mg of N,N-dimethyltryptamine can be administered to a subject in an incremental manner over a period of between 30 minutes and 180 minutes. Most preferably, between 10 mg and 100 mg of N,N-dimethyltryptamine can be administered to a subject in an incremental manner over a period of between 60 minutes and 120 minutes. By incremental manner it is understood herein that between 10 mg and 100 mg of N,N-dimethyltryptamine is administered in smaller portions every time interval, the time interval preferably being selected from a range between 5 minutes and 30 minutes. As described in Example 2, N,N-dimethyltryptamine can be administered to a subject in portions of 5 mg every 15 minutes for a period of 120 minutes, with additional administration of 10 mg at the beginning of the process. Herein, administration is performed by hubs from intranasal spray, each hub corresponds to 2.5 mg of N,N-dimethyltryptamine fumarate. This way, 50 mg of N,N-dimethyltryptamine fumarate are administered to a subject within a period of 120 minutes. It is to be noted that this dosing regime is given for example purposes only, the skilled person will understand that different incremental dosing regimes of N,N-dimethyltryptamine are possible according to the present invention. It is further noted that incremental administration of N,N-dimethyltryptamine is not limited to intranasal administration, other administration routes are possible according to the present invention. In certain preferred embodiments, between 60 mg and 120 mg of N,N-dimethyltryptamine can be administered to a subject in an incremental manner over a period of between 60 minutes and 120 minutes.

Sustained release formulations comprising (a) can also be used in the present invention. Thus, similar dosing regime as outline above can be achieved by applying the sustained release formulation that is administered once but gradually releases 10 mg and 100 mg of N,N-dimethyltryptamine over a certain period of time, preferably less than 240 minutes (4 hours).

Preferably, the sustained release formulation gradually releases between 10 mg and 100 mg of N,N-dimethyltryptamine to a subject over a period of between 30 minutes and 180 minutes. Most preferably, the sustained release formulation gradually releases between 10 mg and 100 mg of N,N-dimethyltryptamine to a subject over a period of between 60 minutes and 120 minutes. Sustained release formulations are known to the skilled person and include for example capsules obtained using micro-encapsulation process and configured for a sustained gradual release of an active ingredient over a certain period of time. Preferably, the sustained release formulations for the use in the present invention include micropellets, minitablets, matrix tablets, or osmotic release oral system.

In the present invention, the inventive composition or parts of the inventive kit of parts are preferably to be administered to a subject in need there of two or three times per week for a duration of preferably between one and four weeks, more preferably for a duration of two weeks. Such a treatment schedule is referred to as treatment block. According to the present invention, there are intervals between the treatment blocks of preferably between 2 and 12 weeks, more preferably of between 6 and 8 weeks. Depending on the needs of the subject (e.g. severity and type of the disorder), the treatment intervals can vary based on judgment of a skilled person. Depending on the type of the disorder (e.g. seasonal, episodic, recurrent, stress-related etc.), intervals between treatment blocks can be extended beyond the above mentioned periods (e.g. 1-2 years).

Further aspects and/or embodiments of the invention are disclosed in the following numbered items:
1. A kit of parts comprising:
   (a) N,N-dimethyltryptamine or a pharmaceutically acceptable salt thereof, and a pharmaceutically acceptable carrier; and
   (b) harmine or a pharmaceutically acceptable salt thereof, and a pharmaceutically acceptable carrier.
2. A composition comprising N,N-dimethyltryptamine fumarate and harmine hydrochloride.
3. A pharmaceutical composition comprising:
   (a) N,N-dimethyltryptamine or a pharmaceutically acceptable salt thereof, and a pharmaceutically acceptable carrier; and
   (b) harmine or a pharmaceutically acceptable salt thereof, and a pharmaceutically acceptable carrier.
4. The kit of parts according to item 1 or the pharmaceutical composition of item 3, wherein (a) is N,N-dimethyltryptamine fumarate and a pharmaceutically acceptable carrier.
5. The kit of parts according to item 1 or 4, or the pharmaceutical composition of item 3 or 4, wherein (b) is harmine hydrochloride and a pharmaceutically acceptable carrier.
6. The kit of parts according to any one of items 1, 4 or 5, or the kit of parts of any one of items 3, 4 or 5, for use as a medicament.
7. The kit of parts for use or the pharmaceutical composition for use according to item 6, for use in treating a psychiatric, psychosomatic or somatic disorder.
8. The kit of parts for use or the pharmaceutical composition for use according to item 6 or 7, wherein the psychiatric disorder is depression, stress-related affective disorder, major depressive disorder, dysthymia, treatment-resistant depression, burnout, anxiety, post-traumatic stress disorder, addiction, eating disorder, or obsessive-compulsive disorder.
9. The kit of parts for use or the pharmaceutical composition for use according to any one of items 6 to 8, wherein (a) and (b) are not to be administered perorally.
10. The kit of parts for use or the pharmaceutical composition for use according to any one of items 6 to 9, wherein (a) and (b) are to be administered simultaneously or sequentially.
11. The kit of parts for use or the pharmaceutical composition for use according to any one of items 6 to 10, wherein (a) is to be administered intranasally.
12. The kit of parts for use or the pharmaceutical composition for use according to any one of items 6 to 11, wherein (a) is to be administered in a dose of between 10 mg to 100 mg of N,N-dimethyltryptamine per administration, preferably in an incremental manner over a period of time of between 60 to 180 minutes.
13. The kit of parts or the pharmaceutical composition for use according to any one of items 6 to 12, wherein (b) is to be administered buccally and/or sublingually.
14. The kit of parts for use or the pharmaceutical composition for use according to any one of items 6 to 13, wherein (b) is to be administered in a dose of between 75 mg to 300 mg of harmine per administration.
15. The kit of parts for use or the pharmaceutical composition for use according to any one of items 6 to 14, wherein the administration of (b) is to be followed by the administration of (a).
16. A method of treating a psychiatric, psychosomatic or somatic disorder, comprising administering to a subject in need thereof:
   (a) N,N-dimethyltryptamine or a pharmaceutically acceptable salt thereof, and a pharmaceutically acceptable carrier; and
   (b) harmine or a pharmaceutically acceptable salt thereof, and a pharmaceutically acceptable carrier;
   wherein (a) and (b) are administered simultaneously or sequentially.
17. The method according to item 16, wherein (a) is N,N-dimethyltryptamine fumarate and a pharmaceutically acceptable carrier.
18. The method according to item 16 or 17, wherein (b) is harmine hydrochloride and a pharmaceutically acceptable carrier.
19. The method according to any one of items 16 to 18, wherein (a) and (b) are not administered perorally.
20. The method according to any one of items 16 to 19, wherein (a) is administered intranasally.
21. The method according to any one of items 16 to 20, wherein (a) is administered in a dose of between 10 mg to 100 mg of N,N-dimethyltryptamine per administration, preferably in an incremental manner over a period of between 60 to 180 minutes.
22. The method according to any one of items 16 to 21, wherein (b) is administered buccally and/or sublingually.
23. The method according to any one of items 16 to 22, wherein (b) is administered in a dose of between 75 mg to 300 mg of harmine per administration.
24. The method according to any one of items 16 to 23, wherein the administration of (b) is followed by the administration of (a).
25. The method according to any one of items 16 to 24, wherein the psychiatric disorder is depression, stress-related affective disorder, major depressive disorder, dysthymia, treatment-resistant depression, burnout, anxiety, post-traumatic stress disorder, addiction, eating disorder, or obsessive-compulsive disorder.

### REFERENCES

Barbosa, P. C. R. et al., 2012. Health status of ayahuasca users. S. D. Brandt & T. Passie, eds. Drug Testing and Analysis, 4(7-8), pp. 601-609.
Barker, S. A., 2018. N, N-Dimethyltryptamine (DMT), an Endogenous Hallucinogen: Past, Present, and Future Research to Determine Its Role and Function. Frontiers in neuroscience, 12, pp. 139-17.
Callaway, J. C. et al., 1996. Quantitation of N,N-dimethyltryptamine and harmala alkaloids in human plasma after oral dosing with ayahuasca. Journal of analytical toxicology, 20(6), pp. 492-497.
Domínguez-Clavé, E. et al., 2016. Ayahuasca: Pharmacology, neuroscience and therapeutic potential. Brain research bulletin.
Frecska, E., Bokor, P. & Winkelman, M., 2016. The Therapeutic Potentials of Ayahuasca: Possible Effects against Various Diseases of Civilization. Frontiers in pharmacology, 7(e42421), pp. 35-17.
Nichols, D. E., 2016. Psychedelics. Pharmacological Reviews, 68(2), pp. 264-355.
Osório, F. de L. et al., 2015. Antidepressant effects of a single dose of ayahuasca in patients with recurrent depression: a preliminary report., 37(1), pp. 13-20.
Palhano-Fontes, F. et al., 2018. Rapid antidepressant effects of the psychedelic ayahuasca in treatment-resistant depression: a randomized placebo-controlled trial. Psychological medicine, 7, pp. 1-9.
Riba, J. et al., 2003. Human pharmacology of ayahuasca: subjective and cardiovascular effects, monoamine metabolite excretion, and pharmacokinetics. The Journal of pharmacology and experimental therapeutics, 306(1), pp. 73-83.
Riba, J. et al., 2015. Metabolism and urinary disposition of N,N-dimethyltryptamine after oral and smoked administration: a comparative study. Drug Testing and Analysis, 7(5), pp. 401-406.
Sanacora, G. et al., 2016. Balancing the Promise and Risks of Ketamine Treatment for Mood Disorders.
Santos, Dos, R. G. et al., 2016. Antidepressive and anxiolytic effects of ayahuasca: a systematic literature review of animal and human studies., 38(1), pp. 65-72.
Schuler et al., 2016. Psychische Gesundheit in der Schweiz. Neuchatel Schweizerisches Gesundheitsobservatorium., pp. 1-80.
Strassman, R. J. et al., 1994. Dose-response study of N,N-dimethyltryptamine in humans. II. Subjective effects and preliminary results of a new rating scale. Archives of general psychiatry, 51(2), pp. 98-108.
Studerus, E., Gamma, A. & Vollenweider, F. X., 2010. Psychometric evaluation of the altered states of consciousness rating scale (OAV). PloS one, 5(8), p. e12412.

### BRIEF DESCRIPTION OF FIGURES

FIG. 1 shows PK profiles of harmine and DMT, when given both orally (single administration; upper panel; day 1) or when harmine is given buccally with incremental intranasal DMT application (lower panel; day 3).

FIG. 2 depicts representative PK profiles of an extensive metabolizer (dotted line) and a poor metabolizer (continuous line) for day 1 (peroral pharmahuasca; upper panel) and day 3 (parenteral pharmahuasca; lower panel).

FIG. 3 shows subjective intensity and valence (liking) ratings for day 1 (peroral pharmahuasca; part 1) and day 3 (parenteral pharmahuasca; part 2).

FIG. 4 summarizes undesired side effects ratings during day 1 (peroral pharmahuasca; left panel) and day 3 (parenteral pharmahuasca; right panel).

FIG. 5 depicts time courses of systolic (circle) and diastolic (triangle) blood pressure, pulse (square) and body temperature (lower panel) during day 1 (peroral pharmahuasca; left panel) and day 3 (parenteral pharmahuasca; right panel).

FIG. 6A depicts ratings in the altered states of consciousness questionnaire (5D-ASC) during day 1 (peroral pharmahuasca; dotted line) and day 3 (parenteral pharmahuasca; continuous line).

FIG. 6B depicts subdimensions of the altered states of consciousness questionnaire (5D-ASC) during day 1 (peroral pharmahuasca; dotted line) and day 3 (parenteral pharmahuasca; continuous line).

FIG. 7 depicts a representative PK profile of a single participant that discontinued intranasal DMT administration at T90 on study day 3, which was followed by an immediate drop in DMT plasma concentrations underscoring the safety and flexibility of sequential dosing.

FIG. 8 depicts a hypothesized model of the biphasic action of peroral pharmahuasca (upper panel A) with an initial psychedelic phase that is driven by fast DMT absorption into the blood stream. With sequential-incremental DMT administration (parenteral pharmahuasca, lower panel B) the distressing aspects of the initial psychedelic phase (e.g. visual hallucinations, perceptive distortions, derealisation, or confusion) can be attenuated, while preserving the empathogenic-entactogenic effects of the drug (e.g. intensified emotions, enhanced introspection, compassion, affective connectedness), which increases the overall safety and tolerability for psychedelic-assisted therapy in patient populations.

FIG. 9 depicts comparison of plasma concentrations of harmine and DMT in two subjects—one that responded to treatment with DMT/harmine, and one that did not respond.

FIG. 10 shows individual responses of subjects administered with peroral pharmahuasca (Part 1 and 2) at different time points of day 1 and day 2 of the study, assessing their experience regarding: A) overall intensity, B) liking of the effects, C) arousal and D) body boundaries. Part 3 of the Figure shows subjective effects with parenteral pharmahuasca in the same study population, assessing their experience regarding overall intensity, liking, arousal, and relaxation at study day 3.

FIG. 11 depicts side effects as assessed by subjects using peroral pharmahuasca in the course of day 1 of the study, regarding A) nausea, B) somatic distress and C) psychological distress (part 1). The Part 3 of the Figure shows the side effect profile of parenteral pharmahuasca in the same population of subjects at study day 3.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention which is defined by the appended claims.

### EXAMPLES

Participants and Permission: N=10 healthy male subjects (20-40 years, mean age 30.7±5.4 years; Body Mass Index of between 18.5 and 25) with no current use of drugs or medications, no current or previous history of somatic, neurological or psychiatric disorder and no family history of Axis-I psychiatric disorder were recruited by medical screening. The study was approved by the Cantonal Ethics Committee of the Canton of Zurich (Basec-Nr. 2018-01385) and Swiss Federal Office of Public Health (BAG-Nr. (AB)-8/5-BetmG-2019/009268). All participants provided written informed consent according to the declaration of Helsinki and were monetary compensated for the completion of the study.

Study setting: The study was conducted during the daytime in soundproof, climatized, and furnished bedrooms to provide a comfortable living room atmosphere with dimmable lights and sound systems. Throughout all study days, a standardized playlist containing non-stimulating background music was played to provide a feeling of comfort and relaxation. An experimenter was present in the room all the time to supervise the participants.

Study design: In this open-label, dose-finding, pilot study, acute subjective effects and blood samples following the administration of 250 mg harmine with a dose of 30 mg vs. 50 mg of DMT (single peroral administration—Example 1) vs. 150 mg buccal harmine with 50 mg of sequential intranasal DMT administration (Example 2) have been tested. On all study days, harmine was premedicated 30 min prior to DMT administration. As this was a dose-finding study, participants were given preferable dose ranges (e.g. 50 mg for day 3) and could dis-/continue further dose administration within the indicated margins (e.g. 0-5 mg of DMT every 15 mins over 120 mins) to enhance safety and tolerability.

Blood sampling and analysis: Blood samples for analysis of DMT and harmine concentrations in plasma were collected from the left antecubital vein at −30 (baseline), −15, 0, 15, 30, 45, 60, 75, 90, 120, 180, 240, 300, and 360 min (peroral pharmahuasca study) and at −30 (baseline), −15, 0, 15, 30, 45, 60, 75, 90, 105, 120, 135, 180, 240 and 300 min after drug administration (parenteral pharmahuasca study). The venous catheter was connected to Heidelberger plastic tube extensions, to collect blood samples without disturbing the subjects during their psychedelic experience. The intravenous line was kept patent with a slow drip (10 ml/h) of heparinized saline (1000 RJ heparin in 0.9 g NaCl/dL; HEPARIN Bichsel; Bichsel AG, 3800 Unterseen, Switzerland). Blood samples were immediately centrifuged for 10 minutes at 2000 RCF and plasma samples were transferred to Eppendorf tubes, shock-frozen in liquid nitrogen (˜−196° C.) and stored at −80° C. until assay. For analytic purposes, DMT was purchased from Cayman (Ann Arbor, USA), and harmine was purchased from Sigma-Aldrich (St. Louis, USA) and DMT-d6 were purchased from Toronto Research Chemicals (Toronto, Canada). All other used chemicals were of highest grade available. For the sample preparation 200 μl of plasma, 50 μl of the internal standard (IS) (20 ng/ml DMT-d6) and 50 μl of Methanol (MeOH) were added to a tube. Proteins were precipitated by adding 400 μl of acetonitrile (ACN) and samples were shaken for 10 minutes and centrifuged for 5 min at 2000 RCF. 350 μl of the supernatant was further transferred into an auto-sampler vial, evaporated to dryness under a gentle stream of nitrogen and reconstituted with 250 μl of an eluent-mixture (98:2, v/v). Calibrator and quality control (QC) samples were prepared accordingly, replacing the MeOH with calibrator or QC solutions. Samples were analyzed on an ultra-high performance liquid chromatography (UHPLC) system (Thermo Fisher, San Jose, Calif.) coupled to a linear ion trap quadrupole mass spectrometer 5500 (Sciex, Darmstadt, Germany). The mobile phases consisted of a mixture of water (eluent A) and ACN (eluent B), both containing 01% of formic acid (v/v). Using a Kinetex C18 column (100×2.1 mm, 1.7 μm) (Phenomenex, Aschaffenburg, Germany), the flow rate was set to 0.5 mL/min with the following gradient: start conditions 98% of eluent A for 0.8 min, decreasing to 60% within 6.7 min followed by a quick decrease to 8% within 0.1 min. These conditions were held for 0.9 min and switched to the starting conditions for re-equilibration for 0.5 min. The mass spectrometer was operated in positive electrospray ionization mode with scheduled multiple reaction monitoring. The following transitions of precursor ions to product ions were selected: DMT, m/z 189.1->58.2, DMT-D3, m/z, 195.1->64.1, harmine, m/z 213.0->169.2. The concentration range in calibration standards was 0.5 ng/ml to 60 ng/ml for DMT, and 3 ng/ml to 360 ng/ml for harmine. Thus, the lower limit of sensitivity was 0.5 mg/ml for DMT, and 3 ng/ml for harmine.

Psychometry: The intensity and valence of subjective effects was monitored throughout the study with visual analogue scales (VAS range 0-100 on a touchscreen tablet) at baseline, −15, 0, 15, 30, 45, 60, 90, 120, 180, 240, and 360 min (peroral pharmahuasca study) and at baseline, −15, 0, 15, 30, 45, 60, 75, 90, 105, 120, 135, 180, 240, and 300 min after drug administration (parenteral pharmahuasca study). Additionally, the Altered States of Consciousness Rating Scale (5D-ASC) (Studerus et al. 2010) was included. The quantitative psychometric assessments were complemented by semi-structured qualitative interviews that were audio-recorded towards the end of the experimental day with a focus on the phenomenology of what people report about their experience.

Vital signs and adverse effects: The participants were screened for (serious) adverse effects throughout the experiment by the study physician, including questionnaire-based assessments (visual analogue scales, 0-100 or yes/no) of physical and mental discomfort, breathing difficulties, racing heartbeat, chest or abdominal pains, unpleasant body sensations/muscle pains, headache, nausea, vomiting, and fainting at baseline, 60, 120, 180, and 240 min after drug administration. Vital signs (systolic/diastolic blood pressure, heart rate, body temperature) were monitored throughout the study at −30 (baseline), 30, 90, 150, 210, and 360 min (peroral pharmahuasca study) and at −30 (baseline), 0, 30, 60, 90, 120, 180, 240, and 300 min after drug administration (parenteral pharmahuasca study).

Study drug: DMT fumarate was obtained as follows: DMT in its basic form was obtained by acidic-basic aqueous extraction from the root bark of Mimosa hostilis (The Mimosa Company, 1069CL Amsterdam, NL), with n-heptane as organic solvent DMT was purified by crystallisation and further recrystallized as DMT fumarate (CAS: 68677-26-9) via salt precipitation. Briefly, DMT in its basic form was dissolved in acetone (9.82 g of DMT free base in 282 mL of acetone). Fumaric acid was also dissolved in acetone (2.89 g fumaric acid in 414 mL acetone). After that, the fumaric acid solution was slowly added to the DMT solution to form the DMT fumarate salt. The solution was left at room temperature for 60 minutes and crystals of the DMT fumarate salt appeared. Excess acetone was decanted, and the crystals of DMT fumarate were washed twice with 100 mL acetone. The DMT fumarate salt was then dried under vacuum. The final product was subjected to qualitative and quantitative analysis via quantitative Nuclear Magnetic Resonance (qNMR), liquid chromatography-tandem mass spectrometry (LC-MS/MS) and high-performance liquid chromatography (HPLC), revealing a purity of 98.20%±0.37%. Based on the qNMR analysis (performed by Reseachem GmbH, Burgdorf, Switzerland) the study drug is believed to be characterized by DMT to fumarate ratio of about 2:1 (1:0.497). Harmine hydrochloride (Harmine HCl, ≥98%, CAS 343-27-1; C₁₃H₁₂N₂O·HCl; 248.71 g/mol) was procured from Santa Cruz Biotechnology Inc. (Dallas, Tex. 75220, USA).

### Example 1. Peroral Pharmahuasca Study (Reference Example)

Peroral formulation: DMT fumarate (dose for day 1: 30 mg; dose for day 2: 50 mg) was encapsulated into opaque size 0 hydroxypropyl methylcellulose (HPMC; Interdelta S. A. Givisiez, 1762, Switzerland) capsules, whereas mannitol was used as filler. To prolong the duration of MAO inhibition by harmine and thus the half-life of DMT, a harmine formulation with an extended-release (ER) profile was developed. Therefore, 2 types of harmine minitablets were manufactured, either with an immediate release (IR) profile (no retardation) or an extended-release (ER) profile. Then both tablet types were combined in a capsule, to form a combination product, whereas parts of the drug are released immediately (150 mg) and another proportion (100 mg) is released slowly. Harmine IR minitablets were obtained as follows: Harmine HCl was ground and compressed using a Glatt tablet press, to form IR minitablets containing 25 mg of harmine HCl. Harmine ER minitablets were manufactured as follows: Harmine HCl was ground, blended with methocel K and compressed with a Glatt tablet press, to form ER minitablets containing 20 mg harmine HCl. The in vitro dissolution profile was examined according to the European Pharmacopoeia. Harmine minitablets were encapsulated into opaque size 0 HPMC capsules, so that said capsule contained:
5 Harmine HCl ER minitablets (100 mg of harmine in total)—each minitablet including 20 mg of harmine hydrochloride and methocel K, its diameter being substantially equal to 5 mm.
6 Harmine HCl IR minitablets (150 mg of harmine in total)—each minitablet including 25 mg of harmine hydrochloride and HPMN, its diameter being substantially equal to 5 mm.

Dose regimen: During study day 1 and 2, subjects received both harmine and DMT as peroral formulations on empty stomach (last meal >10 hours; last drink >90 mins). 30 minutes following peroral premedication with 250 mg harmine HCl (150 mg immediate release+100 mg extended release), subjects ingested a dose of 30 mg of DMT fumarate on day 1 and 50 mg of DMT fumarate on day 2.

Dose regimen Day 1/2 - combined oral harmine
HCl and oral DMT fumarate administration

| Time | Harmine HCl | Harmine HCl | |
|---|---|---|---|
| [min] | IR [mg] | ER [mg] | DMT fumarate [mg] |
| −30 | 150 | 100 | — |
| 0 | — | — | 30 (Day 1) vs. 50 (Day 2) |

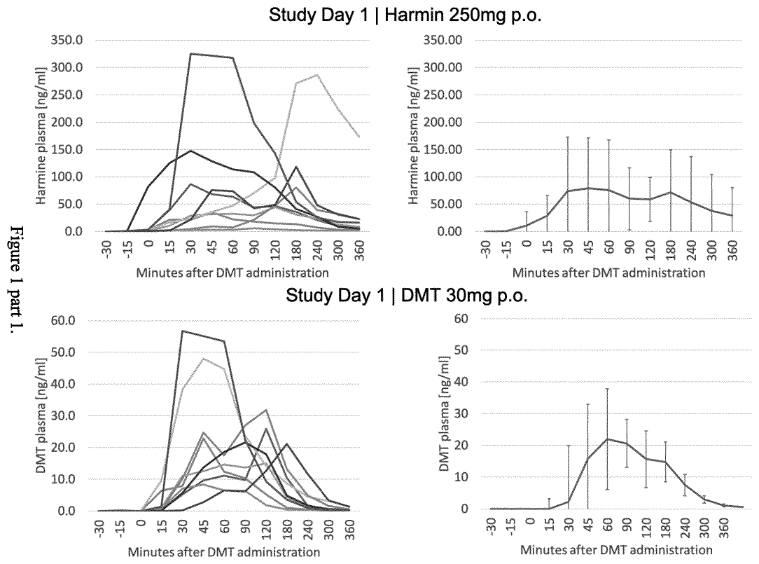

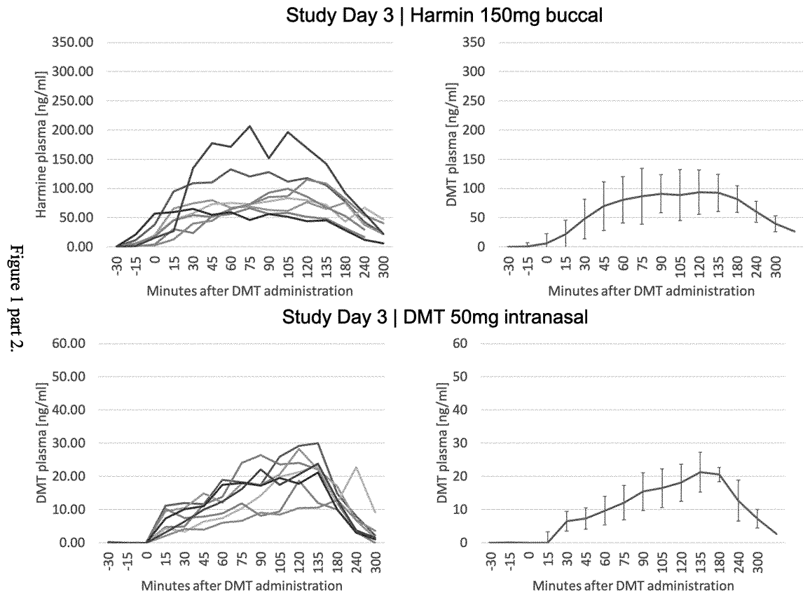

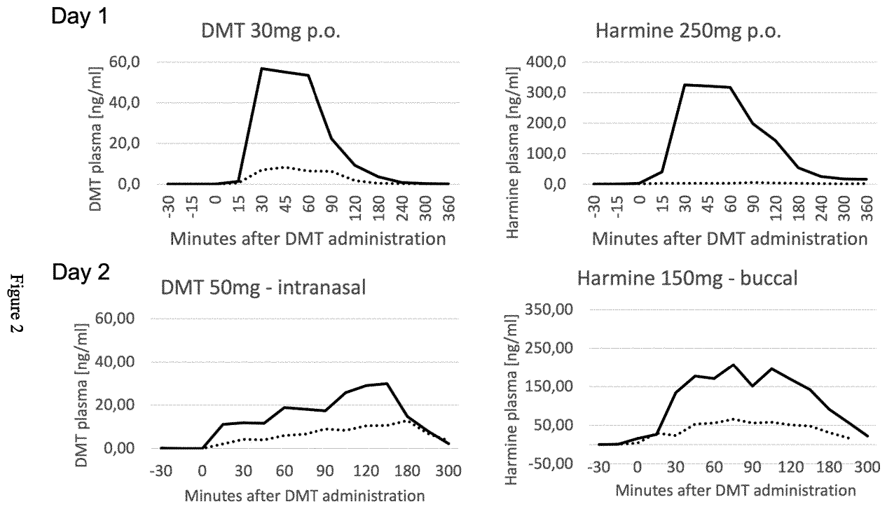

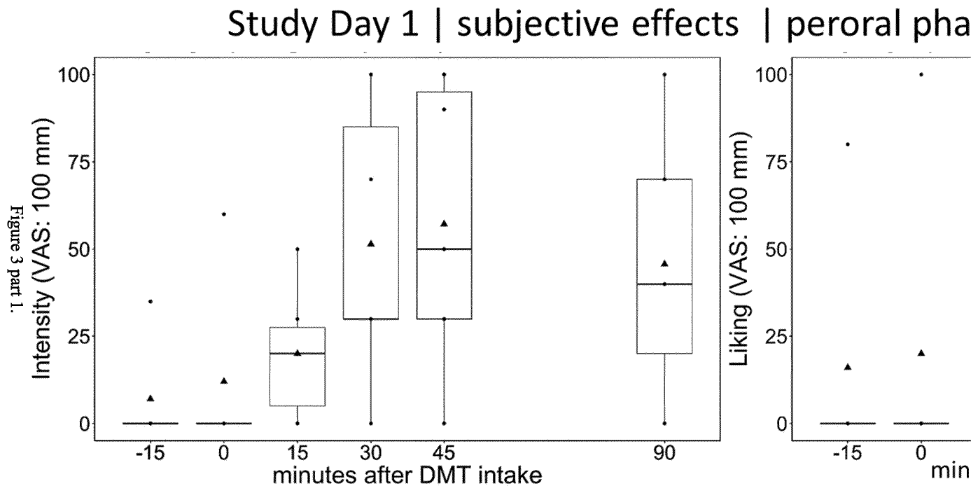

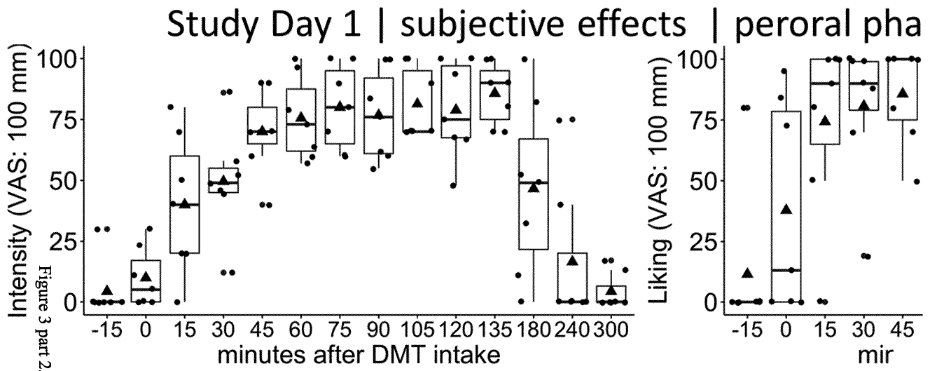

Pharmacokinetic profile: Based on the PK/PD profiles (see FIGS. 1 and 9), it is assumed that the bioavailability and psychotropic potency of DMT depends on the bioavailability of harmine as a MAO inhibitor. Surprisingly, huge interindividual differences in plasma concentrations of both DMT and harmine were found after a single peroral administration (FIG. 9). Peak plasma concentrations (also referred to as Cₘₐₓ) varied substantially by a factor of about 7 for DMT and by a factor of about 50 for harmine across subjects, and likewise time to peak values (tₘₐₓ) of harmine ranged from 60 to 270 minutes (see FIGS. 1 and 2). Accordingly, subjective drug effects varied dramatically between subjects (see FIG. 3), with peak intensity ratings ranging from 1 (non-responder) to 10 (responder). This considerable difference in interindividual responsiveness was highly unexpected and raised doubts, whether peroral pharmahuasca would be clinically applicable.

Harmine is mainly degraded by the hepatic enzyme CYP2D6. Multiple allelic variants of the CYP2D6 gene have been identified, which are associated with a reduced or increased enzyme activity in individuals who are respectively so-called poor (PMs), extensive metabolizers (EMs) and ultrarapid metabolizers (UMs) (Peñas-Lledõ & Llerena, 2014). Thus, depending on an individual's allelic variant of the CYP2D6 gene, the bioavailability of harmine may vary substantially across subjects. Based on the diet recommendations established in the context of traditional ayahuasca ceremonies (e.g. low-tyramine diet) and studies on different metabolism rates (CYP2D6) by harmine, it is concluded that the MAO enzyme activity in the GI tract is subject to considerable inter-individual variability and a reliable standardization of the DMT effect can only be achieved by parenteral administration. The heterogeneity and variability of the transient psychotropic DMT effect following peroral administration necessitates a change in galenic formulation and dosing regimen that produces more robust and sustainable neurobehavioral effects.

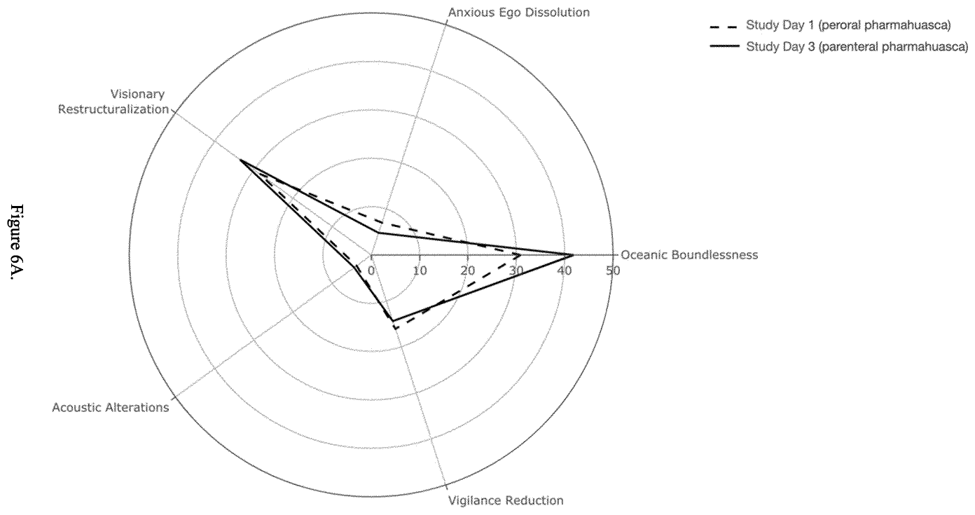

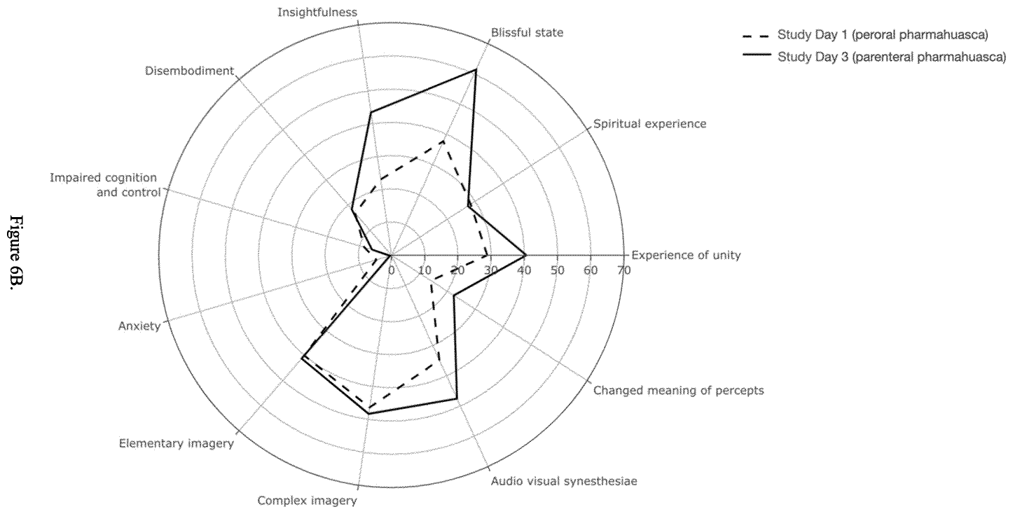

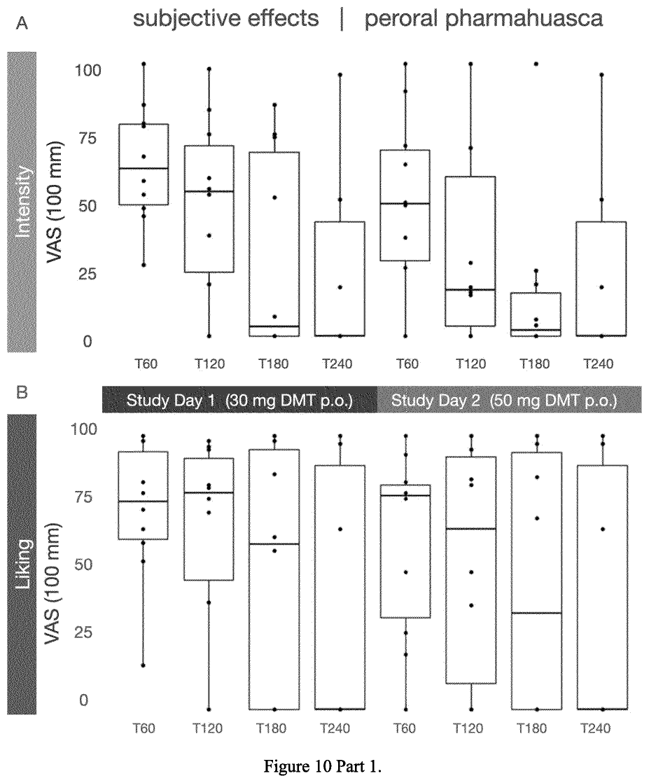

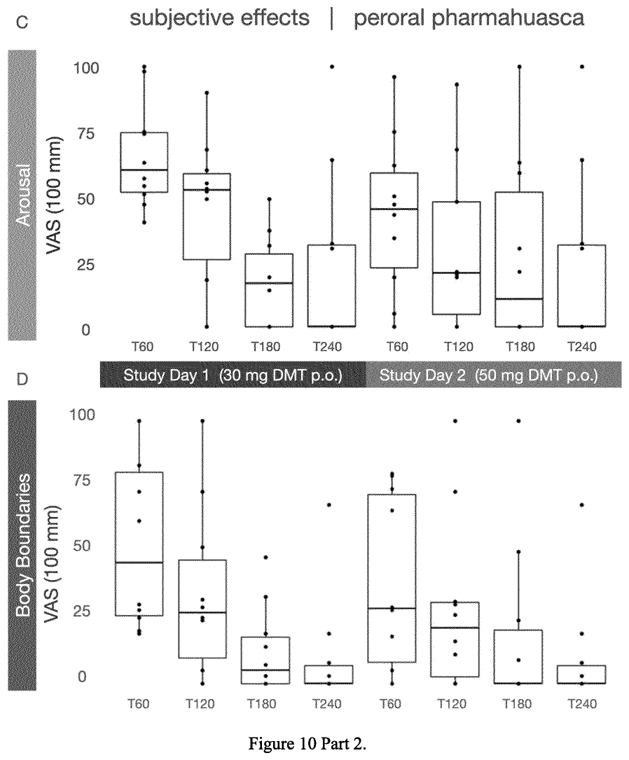

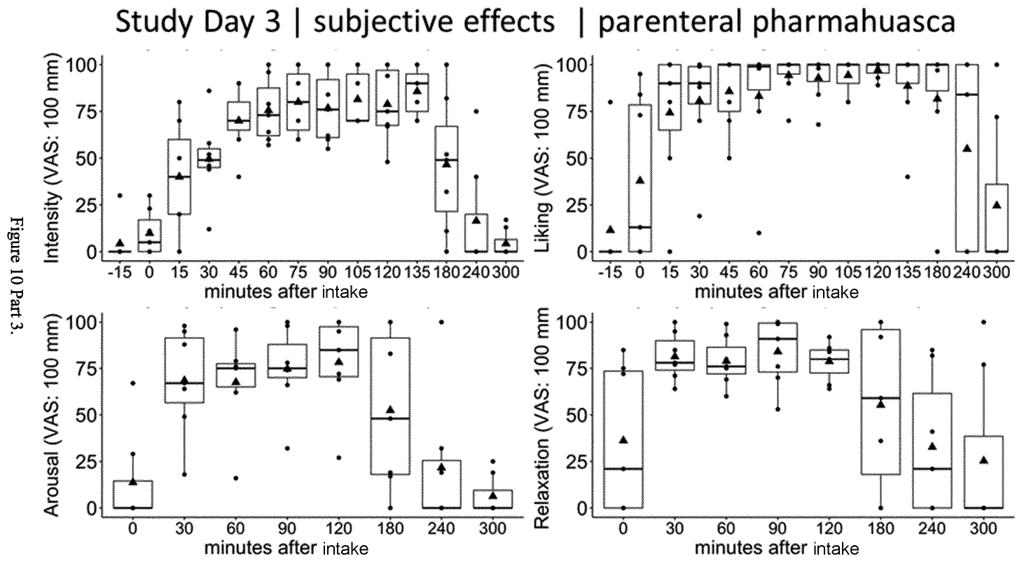

Psychometric assessment: The peroral administration of 250 mg harmine with 30 mg DMT was on average well tolerated. However, the psychotropic effects in comparison to traditional ayahuasca were only transient and significantly less pronounced (4 non-responders without psychotropic effects, 3 partial responders with only short-term DMT effects, 3 responders with typical and briefly overwhelmingly intense DMT effects; FIG. 3). Surprisingly, no marked differences in VAS ratings (e.g. overall intensity, liking, arousal, body boundaries) between oral pharmahuasca with a lower (30 mg) vs. a higher dose (50 mg) of DMT (FIG. 10, Part 1 and 2) were found, which underscores the notion that dose-predictability with oral pharmahuasca is very low. The 5D-ASC assessment of altered states of consciousness revealed that peroral pharmahuasca induces transformative experiences (e.g. oceanic boundlessness and visionary restructuralization) that are considered to mediate symptom reduction according to the literature on the efficacy of psychedelic-assisted therapy. No anxious ego-dissolution or acoustic alterations were observed and levels of vigilance reduction were comparably low (FIG. 6A). Qualitative interviews after the experience (data not shown here) confirmed the quantitative psychometric results.

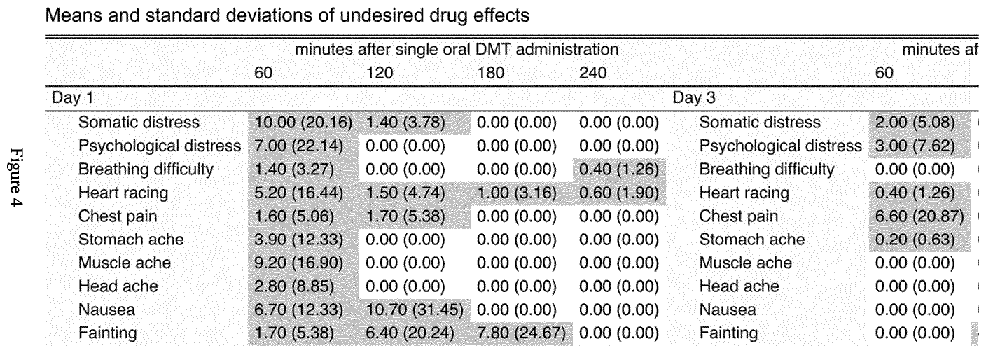

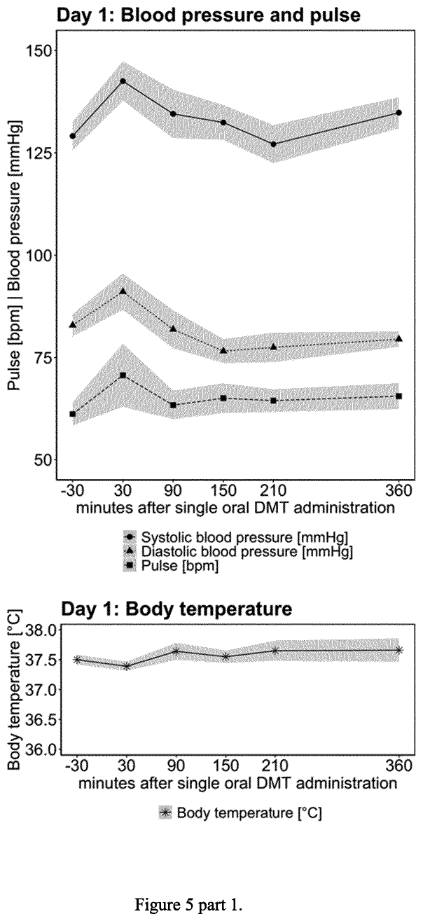

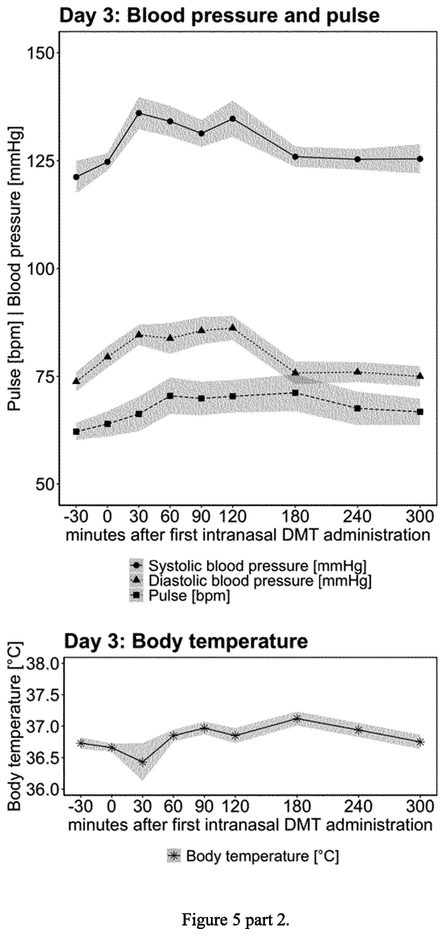

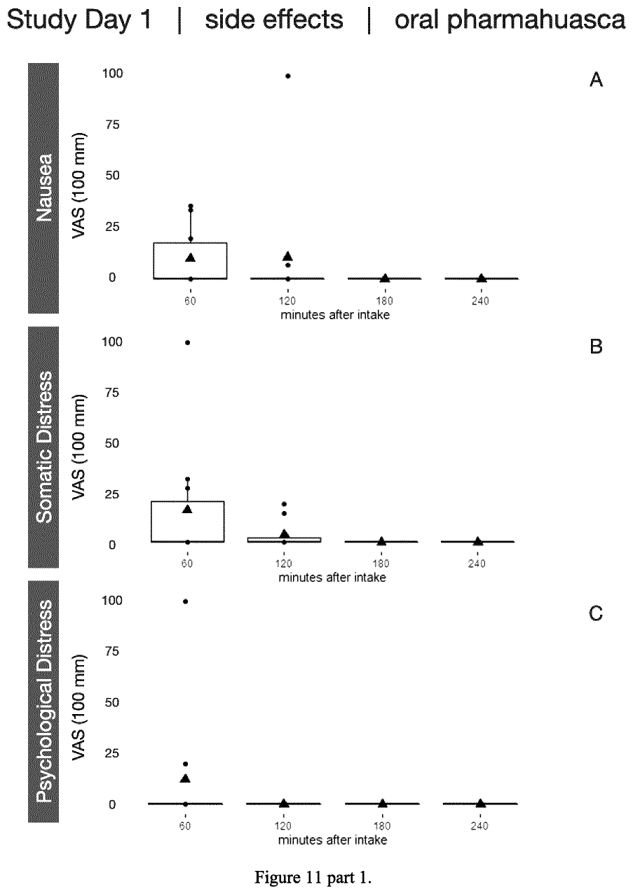

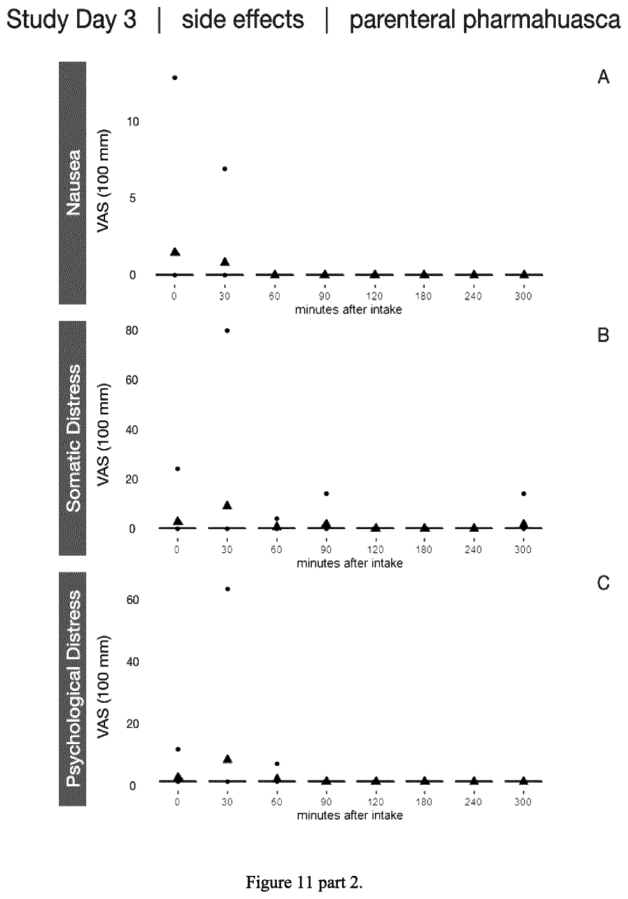

Vital signs and undesired side effects: In comparison with previous studies with traditional ayahuasca, a significantly better tolerability with the peroral preparation described herein (e.g. low spectrum of somatic side effects, especially less nausea and no vomiting) has been demonstrated (FIG. 4; FIG. 11, Part 1). In general, the administration of peroral pharmahuasca induced only transient, asymptomatic, and clinically non-significant elevations of cardiovascular parameters and body temperature (FIG. 5), which underscores an overall very good safety profile.

### Example 2. Combined Intranasal/Buccal Study (Parenteral Pharmahuasca)

Intranasal formulation of DMT fumarate: DMT fumarate nasal sprays were manufactured by dissolving DMT fumarate in saline (0.9 g NaCl/dL) with concentration of 2.5 mg of DMT fumarate per hub. The solution was then transferred into nasal spray PUMP systems with a hub volume of 50 μl (Aptar Pharma, 78431 Louveciennes, France). A total of 50 mg DMT with an added 20% excess volume were prepared to avoid aspiration of air and consequently dilution of the administered dose.

| | | orange | | |
|---|---|---|---|---|
| | DMT fumarate | flavour | | saline (0.9 g NaCl/dL) |
| dose per bottle | 50 mg (+20%) | 28 | mg | add 2.8 ml |
| dose per hub | 2.5 mg | 1.4 | mg | add 0.14 ml |

Sublingual formulation of harmine hydrochloride: Harmine hydrochloride (harmine HCl) orodispersible tablets were manufactured by freeze-drying a harmine/excipient solution. Therefore, harmine HCl (75 mg) was mixed with purified water, mannitol, HPMC and lemon flavor to yield a clear solution. The solution was then volumetrically dosed into aluminum molds, shock-fozen at −80° C. and lyophilized for 36 hours. The final product (75 mg harmine HCl per unit) was then packaged and stored under dry conditions (in the presence of desiccant bags) and dark conditions at room temperature. This formulation is also suitable for use as buccal formulation.

Dose regimen: During study day 3, harmine HCl was administered buccally as orodispersible tablet, while DMT fumarate was applied as intranasal spray solution. 30 minutes following sublingual premedication with 150 mg of harmine HCl (subjects were instructed to keep the tablet between lower lip and gingiva and to avoid excessive swallowing), subjects were administered a cumulative dose of up to 50 mg of DMT fumarate according to the table below. On timepoints 45, 60, 75, 90, 105 and 120 volunteers were allowed to dis-/continue further dose administration within the indicated margins (e.g. 0-5 mg of DMT every 15 mins) to enhance safety and tolerability. Only 6 out of 60 flexible DMT administrations (10%) were skipped by the participants.

Dose regimen Day 3 - combined buccal harmine HCl
and intranasal DMT fumarate administration

| Time [min] | Harmine HCl [mg] | DMT fumarate [mg] |
|---|---|---|
| −30 | 150 | — |
| −15 | — | — |
| 0 | — | 10 |
| 15 | — | 5 |
| 30 | — | 5 |
| 45 | — | 5 |
| 60 | — | 5 |
| 75 | — | 5 |
| 90 | — | 5 |
| 105 | — | 5 |
| 120 | — | 5 |
| Total | 150 | 50 |

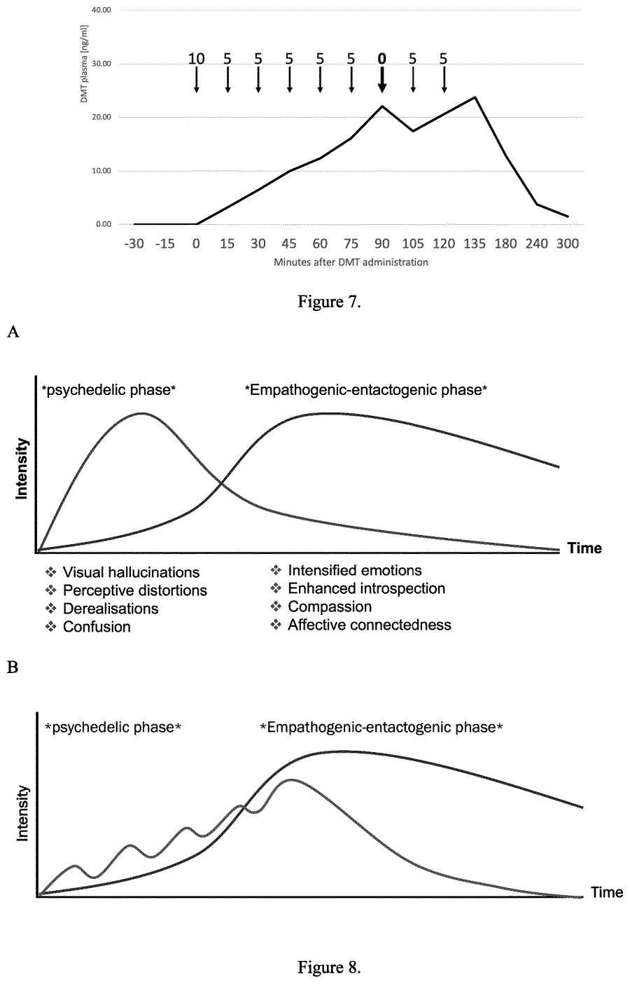

Pharmacokinetic profile: By bypassing the GI tract (first pass metabolism, pH dependence of absorption, GI tract motility etc.) with a parenteral preparation, herein intranasal formulation of DMT fumarate and sublingual formulation of harmine hydrochloride, a better standardization of bioavailability, a lower interindividual variance as well as a more reliable dose-response relationship with presumably fewer somatic side effects can be achieved. Analysis of the blood plasma curves following oral and parenteral administration of DMT/harmine underlines this notion. As shown in FIG. 1, the parenteral administration of both DMT fumarate (intranasal) and harmine HCL (buccal) yield substantially higher bioavailability (higher area under the curve; AUC) and more homogenous plasma curves (reduced standard deviations) compared to their oral administration. Peak plasma concentrations (cₘₐₓ) for extensive vs. poor metabolizers varied by a factor of ˜2.3 for DMT and only a factor of ˜3.2 for harmine across subjects (see FIG. 2). Due to parenteral administration, it is possible to reduce the retarded dose of 250 mg harmine (peroral) to an unretarded dose of 150 mg harmine (buccal) and achieve sufficient CNS inhibition effects. Surprisingly, the buccal delivery of harmine produced a smooth sustained-release profile which is favorable for repeated intermittent dosing of DMT over 120 minutes. Moreover, the time in the therapeutic range (TTR) is significantly longer following the combined intranasal/buccal application of DMT and harmine. Incremental intranasal DMT administration yields a linearly increasing plasma curve with comparably smaller standard deviations, further underlining the superiority of this route of administration compared to the oral route, namely in terms of predictability and reliability. In addition, intranasal DMT administration for dose determination offers a better safety profile due to increased controllability, because DMT doses can be administered incrementally and sequentially. The pharmacological intervention can be interrupted at any time if side effects occur, which is not the case with peroral administration of a retarded DMT preparation. FIG. 7 shows a representative PK profile of a single participant that discontinued intranasal DMT administration at T90, which was followed by an immediate drop in DMT plasma concentrations without affecting subsequent DMT administrations at T105 and T120. This example highlights the flexibility of the intranasal sequential-intermittent DMT dosing regimen for improving the safety in patient populations, given the need to minimize the psychological risks inherent with the use of psychedelic compounds.

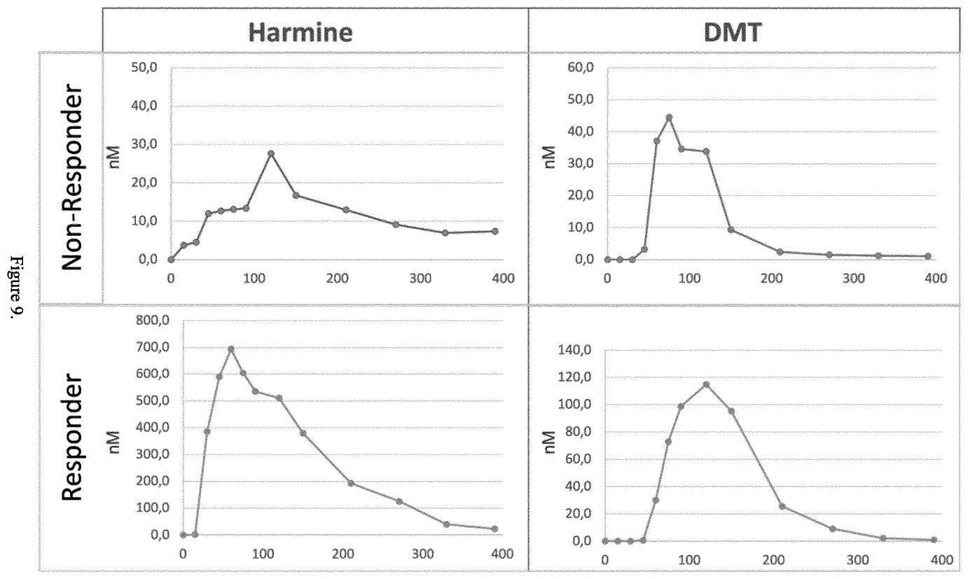

Psychometric assessment: The sublingual administration of 150 mg harmine with 50 mg DMT (intranasally) was very well tolerated (low scores on anxiety, loss of control, disorientation; VAS ratings of “liking” between 70-100 out of 100; see FIG. 3, Part 2 and FIG. 10, Part 3). Compared to the oral route of administration, the psychotropic effects were much more pronounced and sustained, resulting in an improved response predictability (9 out of 10 subjects fully responded until 180 min after DMT administration; see FIG. 3, Part 2). Compared to peroral pharmahuasca, the 5D-ASC ratings revealed higher levels of oceanic boundlessness and visionary restructuralization and lower levels of vigilance reduction with parenteral pharmahuasca (FIG. 6A). On the level of the subscales, an optimized experience profile with higher levels of transformative experiences (e.g. blissful state, experience of unity, and insightfulness) could be achieved, with very low levels of anxiety, disembodiment, or impaired cognition and control (FIG. 6B). Such an experience profile is unique for a psychedelic agent and is presumably related to the optimized galenic of DMT and harmine co-administration (see also FIG. 8). Compared to other psychedelics, parenteral pharmahuasca does not impair cognition and does not induce anxiety or confusion and hence seems to be very well suited to support psychotherapy. Based on qualitative interviews, study participants clearly favored the parenteral over the oral formula. Most importantly, the incremental administration of DMT permitted to shape the dosing regimen according to participant's feedback, making it much more controllable and thus safer to guide the participants through the psychedelic experience. Participants have also noted that the incremental administration of DMT attenuates the initial distressing aspects of the psychedelic experience while preserving the empathogenic properties of the drug which is more favorable for clinical applications in vulnerable patient populations (for details see FIG. 8).

Vital signs and undesired side effects: Compared to the oral administration, on average less somatic side effects (e.g. nausea, distress) occurred with parenteral administration, indicating a better safety and tolerability profile (FIG. 11, Part 2). While vomiting and diarrhea are common features of traditional ayahuasca, no such cases were observed in our study sample. Further details are shown in FIGS. 4 and 5.

1. A kit of parts comprising:
(a) N,N-dimethyltryptamine or a pharmaceutically acceptable salt thereof, and a pharmaceutically acceptable carrier; and
(b) harmine or a pharmaceutically acceptable salt thereof, and a pharmaceutically acceptable carrier.

2. A composition comprising N,N-dimethyltryptamine fumarate and harmine hydrochloride.

3. A pharmaceutical composition comprising:
(a) N,N-dimethyltryptamine or a pharmaceutically acceptable salt thereof, and a pharmaceutically acceptable carrier; and
(b) harmine or a pharmaceutically acceptable salt thereof, and a pharmaceutically acceptable carrier.

4. The kit of parts according to claim 1 or the pharmaceutical composition of claim 3, wherein (a) is N,N-dimethyltryptamine fumarate and a pharmaceutically acceptable carrier.

5. The kit of parts according to claim 1 or 4, or the pharmaceutical composition of claim 3 or 4, wherein (b) is harmine hydrochloride and a pharmaceutically acceptable carrier.

6. The kit of parts according to any one of claim 1, 4 or 5, or the kit of parts of any one of claim 3, 4 or 5, for use as a medicament.

7. The kit of parts for use or the pharmaceutical composition for use according to claim 6, for use in treating a psychiatric, psychosomatic or somatic disorder.

8. The kit of parts for use or the pharmaceutical composition for use according to claim 6 or 7, wherein the psychiatric disorder is depression, stress-related affective disorder, major depressive disorder, dysthymia, treatment-resistant depression, burnout, anxiety, post-traumatic stress disorder, addiction, eating disorder, or obsessive-compulsive disorder.

9. The kit of parts for use or the pharmaceutical composition for use according to any one of claims 6 to 8, wherein (a) and (b) are not to be administered perorally.

10. The kit of parts for use or the pharmaceutical composition for use according to any one of claims 6 to 9, wherein (a) and (b) are to be administered simultaneously or sequentially.

11. The kit of parts for use or the pharmaceutical composition for use according to any one of claims 6 to 10, wherein (a) is to be administered intranasally.

12. The kit of parts for use or the pharmaceutical composition for use according to any one of claims 6 to 11, wherein (a) is to be administered in a dose of between 10 mg to 100 mg of N,N-dimethyltryptamine per administration, preferably in an incremental manner over a period of time of between 60 to 180 minutes.

13. The kit of parts or the pharmaceutical composition for use according to any one of claims 6 to 12, wherein (b) is to be administered buccally and/or sublingually.

14. The kit of parts for use or the pharmaceutical composition for use according to any one of claims 6 to 13, wherein (b) is to be administered in a dose of between 75 mg to 300 mg of harmine per administration.

15. The kit of parts for use or the pharmaceutical composition for use according to any one of claims 6 to 14, wherein the administration of (b) is to be followed by the administration of (a).
